(19) United States
(12) Patent Application Publication
(10) Pub. No.: US 2023/0248297 A1
(43) Pub. Date: Aug. 10, 2023

(54) SYSTEMS AND METHODS FOR ADMINISTERING A SMELL TEST FOR SARS CORONAVIRUSES AND COVID-19

(71) Applicant: The General Hospital Corporation, Boston, MA (US)

(72) Inventors: Mark William Albers, Winchester, MA (US); Colin Magdamo, Boston, MA (US)

(21) Appl. No.: 17/996,911

(22) PCT Filed: Apr. 26, 2021
(86) PCT No.: PCT/US2021/029098

§ 371 Date: Oct. 21, 2022

Related U.S. Application Data
(60) Provisional application No. 63/040,915, filed on Jun. 18, 2020.
(60) Provisional application No. 63/027,921, filed on May 20, 2020.
(60) Provisional application No. 63/014,937, filed on Apr. 24, 2020.

(51) Int. Cl. A61B 5/00; G16H 50/30; G16H 50/80

(52) U.S. Cl. CPC A61B 5/4011; G16H 50/30; G16H 50/80

(57) ABSTRACT

Systems and methods ate provided that may facilitate the self-administration of an odor-based, or smell test. The test may include like patient performing an odor intensity test, odor identity test, and/or odor discrimination test using an odor proctor or testing kit. The testing kit can include the odor proctor and a test guide. The test guide may be used with the odor proctor to perform various smell test(s). The test guide may be realized in software that is readily available via a personal computing device.

### CROSS-REFERENCES TO RELATED APPLICATIONS

The present application represents the U.S. national stage entry of International Application No. PCT/US2021/029098 filed Apr. 26, 2021, which is based on and claims priority to U.S. Provisional Patent Application No. 63/040,915 filed Jun. 18, 2020, entitled “SYSTEMS AND METHODS FOR ADMINISTERING A SMELL TEST FOR SARS CORONAVIRUSES AND COVID-19,” U.S. Provisional Patent Application No. 63/027,921 filed May 20, 2020, entitled “COVID SMELL TEST,” and U.S. Provisional Patent Application No. 63/014,937 filed Apr. 24, 2020, entitled “SMELL TEST FOR SARS CORONAVIRUSES AND COVID-19.” The contents of these applications are incorporated herein by reference in their entirety.

### STATEMENT REGARDING FEDERALLY SPONSORED RESEARCH

Not Applicable.

### BACKGROUND OF THE INVENTION

The world is confronted by a crisis embodied in SARS Coronaviruses, such as SARS-COV-2, and COVID-19, the likes of which have not been seen before. The challenge particular to any pandemic is the ability to control its spread. Control of a pandemic requires information, and that information can only be gleaned by testing. However, an issue that is ubiquitous with testing is that testing, when done on a large scale (i.e., global, countrywide, citywide, etc.), creates substantial logistical challenges. For example, test manufacturing can be a substantial constraint, such as with tests for COVID-19, where reagents and even nasal swabs are in limited supply. Even if the raw materials for manufacturing tests are available, the next logistical hurdle is the need to distribute the tests far and wide, and in large volumes. Even if tests are uniformly available and assuming that trained staff are readily available to administer the test in the volumes required, the next logistical challenge is the need to provide test samples back to a lab for processing. Thus, extensive lab resources are required because delays in securing test results can result in further transmission of the disease.

Further, in the case of a viral pandemic such as COVID-19, the testing itself becomes a mechanism for transmission of the virus. For example, to be tested, a person must self-select to travel to a site to receive the test, which is a location that inherently mixes those that are infected with those that are not yet infected.

Therefore, there is a need for new testing paradigms in the face of such logistical challenges and the creation of substantial transmission vectors presented by traditional testing protocols, such as being utilized in viral pandemics like the COVID-19 pandemic.

### SUMMARY OF THE INVENTION

The disclosure overcomes the aforementioned drawbacks by providing systems and methods that facilitate rapid, self-administered testing of viral infection, such as caused by coronavirus SARS-COV-2, COVID-19. In particular, systems and methods are provided to facilitate the self-administration of an odor-based, or smell test. The test may include other, non-smell or non-odor-based testing or questions. The test may include the patient performing an odor intensity test, odor identity test, and/or odor discrimination test using an odor proctor or testing kit. The testing kit can include the odor proctor and a test guide. The test guide may be used with the odor proctor to perform various smell test(s). The test guide may be realized in software that is readily available via a personal computing device and the odor proctor or test kit can be made ubiquitously available to patients, even by direct supply in volume via mail or the like, which avoids transmission risks. The results can be immediately provided to the patient upon completion of the test, along with instructions for further action based on the results. Smell testing is noninvasive and utilizes, in some cases, an approachable web-based app that can provide a game-like experience, which increases its adaptation among individuals, particularly children, relative to traditional testing methods (i.e., swab based tests). The test may be used not just to diagnose infection, but to track progression of the disease, perform or facilitate risk stratification, provide key care guidance correlated to test results (or longitudinal sets of test results), and even identify the recovery and likely presence of antibodies in the patient.

In some aspects, the present disclosure provides a test kit including an odor proctor configured to deliver an odor from among a plurality of odors and an application on a computing device comprising instructions that, when executed by a processor running on the computing device, can cause the computing device to execute a process. The computing device can generate a user interface to be displayed on the computing device. In some aspects, the computing device can receive, via the user interface, a user input for a smell test comprising at least one of an odor intensity test, an odor identification test, or an odor discrimination test. The computing device can calculate, based on the user input for the smell test, a score for the smell test. In some aspects, the computing device can determine whether the score for the smell test is below a predetermined threshold and responsive to the determination that the score for the smell test is below the predetermined threshold, the computing device can identify a user as high risk for being infected with COVID-19.

In another aspect, the present disclosure provides a method of administering a smell test for assessment of risk of infection by SARS coronaviruses or COVID-19. The method can include generating, by a computing device, a user interface to be displayed on the computing device and instructing, by the computing device via the user interface, a user to utilize an odor proctor configured to deliver an odor from among a plurality of odors. The method can include receiving, by the computing device via the user interface, a user input for a smell test. In some aspects, the method can include calculating, by the computing device, a score for the smell test and based on the score for the smell test, identifying a user as high risk for being infected with SARS coronavirsuses or COVID-19.

The foregoing and other aspects and advantages of the disclosure will appear from the following description. In the description, reference is made to the accompanying drawings which form a part hereof, and in which there is shown by way of illustration a preferred configuration of the disclosure. Such configuration does not necessarily represent the full scope of the disclosure, however, and reference is made therefore to the claims and herein for interpreting the scope of the disclosure.

### BRIEF DESCRIPTION OF THE DRAWINGS

The invention will be better understood and features, aspects and advantages other than those set forth above will become apparent when consideration is given to the following detailed description thereof. Such detailed description makes reference to the following drawings.

FIG. 1 is a schematic illustration of a system for testing subjects in accordance with the present disclosure.

FIG. 2 is a further schematic illustration of a system for testing subjects in conjunction with the system of FIG. 1.

FIG. 3 is a flow diagram setting forth some non-limiting example steps for using the systems in accordance with the present disclosure.

FIG. 4 is a pictorial representation of an example user interface generated by a client or computing device for conducting a smell test in accordance with the present disclosure.

FIG. 5 is a pictorial representation of one example of an odor proctor according to one aspect of the present disclosure.

FIG. 6 is a flow chart setting forth some non-limiting example steps of a method of using a test kit according to some aspect of the present disclosure.

FIG. 7 is a pictorial representation of a patient information interface according to some aspects of the present disclosure.

FIG. 8 is a pictorial representation of a symptom questionnaire interface according to some aspects of the present disclosure.

FIG. 9 is a graphic illustration of instructions for using an odor proctor, such as illustrated in FIG. 5.

FIG. 10 is a pictorial representation of a use interface of an odor test prompt according to one aspect of the present disclosure.

FIG. 11 is a pictorial representation of an odor intensity user interface according to one aspect of the present disclosure.

FIG. 12 is a pictorial representation of an odor identification user interface according to one aspect of the present disclosure.

FIG. 13 is a pictorial representation of an odor discrimination user interface according to one aspect of the present disclosure.

FIG. 14 is a flow chart setting forth steps of an algorithm for identifying a user as having a high risk for COVID-19 infection according to one aspect of the present disclosure.

FIG. 15 is a flow chart setting forth steps of a second algorithm for identifying a user as having a high risk for COVID-19 infection.

FIG. 16 is a graphic illustration of a life cycle of the COVID-19 viral load.

FIG. 17 is a set of graphs showing exemplary results of a research study regarding odor intensity testing in accordance with the present disclosure.

FIG. 18 is a set of graphs showing exemplary results of a research study regarding odor identification testing in accordance with the present disclosure.

FIG. 19 is a set of graphs showing exemplary results of a research study regarding odor discrimination testing in accordance with the present disclosure.

FIG. 20 is a graphic representation of a mechanism of loss of olfactory function according to one aspect of the present disclosure.

FIG. 21 illustrates odds ratios for predicting COVID-19 infection status from results of an exemplary clinical study utilizing the aspects of the present disclosure.

FIG. 22 illustrates sensitivity and specificity of an algorithm utilized in the clinical study.

FIG. 23 illustrates negative predictive value of the algorithm utilized in the clinical study.

### DETAILED DESCRIPTION OF THE INVENTION

The following discussion is presented to enable a person skilled in the art to make and use aspects of the present disclosure. Various modifications to the illustrated configurations will be readily apparent to those skilled in the art, and the generic principles herein can be applied to other configurations and applications without departing from aspects of the present disclosure. Thus, aspects of the present disclosure are not intended to be limited to configurations shown, but are to be accorded the widest scope consistent with the principles and features disclosed herein. The following detailed description is to be read with reference to the figures, in which like elements in different figures have like reference numerals. The figures, which are not necessarily to scale, depict selected configurations and are not intended to limit the scope of the present disclosure. Skilled artisans will recognize the non-limiting examples provided herein have many useful alternatives and fall within the scope of the present disclosure.

In any kind of viral pandemic, such as COVID-19, the virus manifests itself in the form of particular symptoms. These symptoms can provide an excellent way to manage early detection of the virus, or even late detection, including the ability to monitor the progress of the virus (e.g., information that guides care or determining if the virus is still present in a subject). An example of one such symptom is a fever. From the earliest stages of the COVID-19 pandemic, non-contact temperature readings have been used to determine if a subject is infected. However, this method of detection has clear drawbacks. For example, it is well established that fevers do not manifest within a subject suffering from COVID-19 at the early onset of the coronavirus. Thus, an infected subject can travel, visit stores, or otherwise engage with the public for days and even weeks before any fever-based test would alert anyone to the potential infection, accelerating the spread of the virus.

Other symptoms have also been explored for the detection of coronavirus, such as blood clotting and pneumonia. However, these symptoms also do not manifest within a subject at the early onset of the coronavirus. At best, these symptoms are used as tools to help a clinician in the decision making process in a treatment scenario/environment. In other words, these symptoms are, at best, used to determine which treatments a subject should receive, but these symptoms are not useful for early onset detection of coronavirus to help prevent the spread of the virus.

Additionally, at present, the most common and widely accepted method of testing for COVID-19 is the nasopharyngeal swab SARS-COV-2 real time reverse transcription polymerase chain reaction (“NP RT-PCR”) test. However, this test requires a clinician to swab the nasopharynx, which, in addition to the adding to the logistical strain on the healthcare system, is in and of itself a vector for transmission of the virus. In addition, the swabs must be sent to a lab for testing instead of being immediately disposed of, furthering the number of individuals in contact with the swab, which again, can be a mechanism for transmission of the virus. Further, in some cases, not all individuals meet the current criteria for the NP RT-PCR test, meaning that the test is not widely available. Further still, these tests usually take place in testing centers, requiring the gathering of large amounts of people. In addition to all the foregoing drawbacks, the NP RT-PCR test is only about 70% sensitive, meaning that about 30% of “negative” test results are false positive. That is, the patient has the infection, but is informed that they are healthy. Thus, on its own, the NP RT-PCR test is inaccurate. Further, the results of the NP RT-PCR test are not made immediately available to the patient and the patient must wait hours or even days to receive the results.

As will be described in detail herein, the present disclosure provides a test that can be self-administered. The test can include a smell test capable of detecting early stages of an infection, such as COVID-19. The test described herein overcomes all of the drawbacks noted above and provides substantial, additional advantages not otherwise available.

According to aspects of the present disclosure, devices or systems disclosed herein can be utilized, manufactured, or implemented using methods embodying aspects of the invention. Correspondingly, any description herein of particular features, capabilities, or intended purposes of a device or system is generally intended to include disclosure of a method of using such devices for the intended purposes, of a method of otherwise implementing such capabilities, of a method of manufacturing relevant components of such a device or system (or the device or system as a whole), and of a method of installing disclosed (or otherwise known) components to support such purposes or capabilities. Similarly, unless otherwise indicated or limited, discussion herein of any method of manufacturing or using for a particular device or system, including implementing the device or system, is intended to inherently include disclosure, as embodiments of the invention, of the utilized features and implemented capabilities of such device or system.

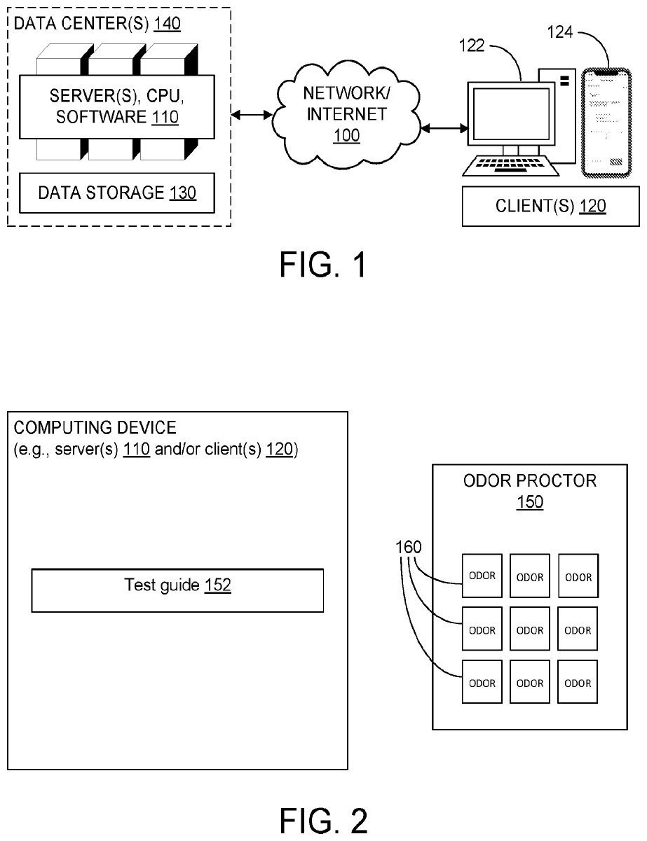

FIGS. 1 and 2 illustrate non-limiting examples of systems that can be used to administer tests and/or determine test results in accordance with the present disclosure. These systems are non-limiting, and other systems are contemplated, including manually administered tests, tests using olfactometers, or other variations on proctors, administration systems, and reporting systems. In the examples provided in FIGS. 1 and 2, one environment may include an odor proctor 150 having at least one odor 160 to be delivered to a subject. In the non-limiting example illustrated in FIG. 2, the odor proctor 150 can be configured to deliver a plurality of odors 160.

In the illustrated configuration, the odor proctor 150 can be configured to be easy to use such that a smell test can be self-administered by the subject. The self-administered nature of the smell test can prevent the spread of the virus by, for example, preventing any interaction with a healthcare worker or by requiring a subject to enter a healthcare facility. That is, the self-administered nature of the smell test, along with the odor proctor 150 described herein, can enable a subject to conduct a smell test in a safe and isolated way (e.g., from within their own residence). For example, a subject may self-administer the smell tests using the odor proctor 150 using, for example, a website or application (e.g., test guide 152) accessible via a client 120 (such as a user's home computer 122 or mobile device 124).

The odor proctor 150 may also be disposable. Further, the odor proctor 150 can be disposed of after the completion of the smell test. For example, after identifying the subject as having a risk (e.g., a “high risk”) for being infected with COVID-19, the client 120 may instruct the subject to dispose the odor proctor 150. In this way, contrary to the current tests being utilized in the art, it is not required to send out the odor proctor 150 (e.g., to a lab or test center) to obtain the results. Thereby further preventing the spread of the virus by eliminating potential transmission vectors. As one non-limiting example, which will be described below, the odor proctor 150 may be or include a printed smell test card. In this non-limiting example, the plurality of odors 160 may be printed on or embedded into the smell test card.

Other configurations of an odor proctor 150 are envisioned as well. For example, the odor proctor 150 may include a collection of encapsulated scents. In some configurations, the scents may be encapsulated in an enclosure and a subject may open the enclosure to be exposed to the scent. Also, the odor proctor 150 may include an olfactometer.

In the non-limiting example illustrated in FIGS. 1 and 2, a subject can use the odor proctor 150 in conjunction with, and guided by, a test guide 152, which may be available via the client 120. The test guide 152 may be realized as software on a client device 120 (i.e., a laptop/desktop computer 122, or tablet or smart phone 124, or any other device capable of operating the software). To this end, the test guide 152 may be distributed as an application installed on a traditional computer 122, an app downloaded to a tablet or smart phone 124, a website accessible by traditional computer 122 or tablet or smart phone 124, or any of a variety of other vehicles, including non-computerized tools, such as booklets or the like.

The test guide 152 may operate using a traditional computer 122 or tablet or smart phone 124 that is operating in isolation or may be part of a networked environment or connected to the Internet 100. One or more processors on a combination of one or more computing devices (e.g., the server computer(s) 110 and/or client devices 120) may interact in a networked environment. For example, the one or more server computers 110 and the client devices 120 can be communicatively coupled to a network 100. The processor(s) may execute instructions (e.g., by compiled and executable software code) that, when executed by the processor, causes the processor to execute the algorithms and method steps described herein for administering the tests and calculating results. The server(s) 110 and/or client(s) 120 may execute the disclosed algorithms to calculate and display the results of the tests to the user/subject and/or communicate the results to a data center 140. Data used for these calculations, as well as the results of these calculations, may be queried from and/or stored within database 130 within the data center 140, which may be communicatively coupled to the network 100, the server 110, and/or the client 120.

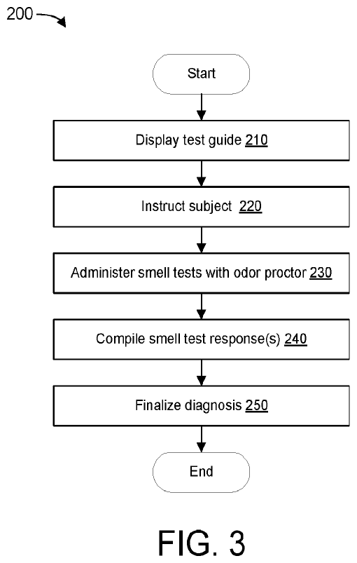

FIG. 3 provides non-limiting and exemplary steps of a method of carrying out a smell test utilizing the odor proctor 150 in conjunction with the test guide 152, thereby forming a test kit. In general, the odor proctor 150 together with the test guide 152 may be used to deliver odors and determine a user, test subject, or patient's ability to discern scents or scent intensities. That is, the odor proctor 150 may be used in conjunction with the test guide, to objectively quantify and qualify human olfaction.

At process block 210, the process begins by using the test guide. The test guide may be pre-printed or may be made available on client(s) as a user interface. The user interface can allow the user/subject/patient to interact with the smell tests and display the results of the smell tests to the subject. As detailed herein, this user interface may include graphical, textual, scanned, and/or auditory information. For example, a computer program or application may present said information to the subject, and monitor/store the control sequences such as keystrokes, movements of the computer mouse, selections with a touch screen, scanned information, and the like used to control the application.

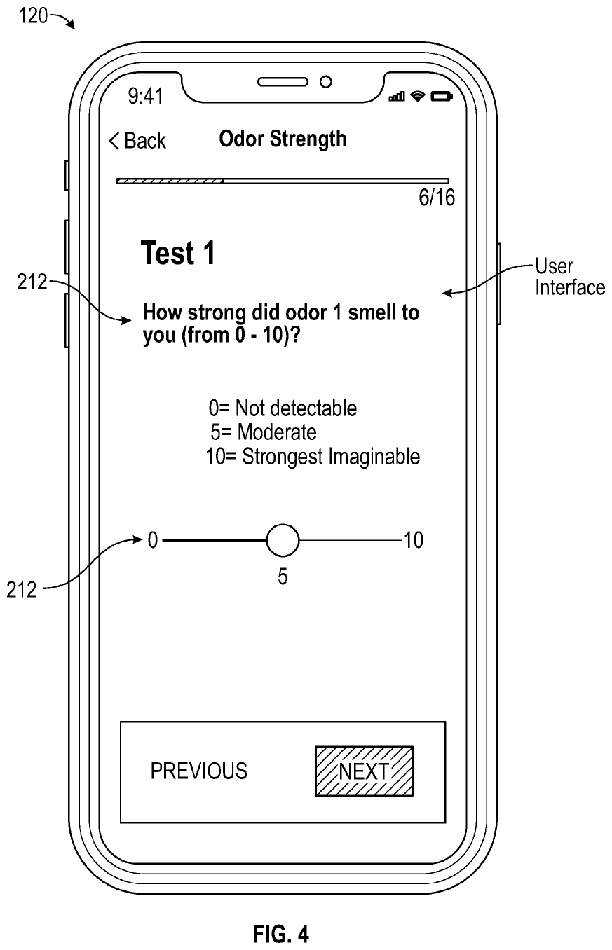

Referring to FIG. 4, one non-limiting example of a user interface with one or more functions 212 generated on a client 120 is shown. The responses/results/commands received within the test guide, or other data, may be accepted or received from the subject using said functions 212. For example, the functions 212 may include a field, widget and/or control used in such user interfaces, including but not limited to a text-box, text field, button, hyper-link, list, drop-down list, check-box, radio button, data grid, icon, graphical image, embedded link, etc. The functions 212 may also include providing smell test instructions to a subject, providing instructions for utilizing the odor proctor 150 of FIG. 2, and/or receiving a user input from a subject based on the smell test.

Referring again to FIG. 3, whether generated on a client 120 or via a different vehicle, test instructions are provided to the subject at process block 220. Upon receiving the test instructions, the subject may interact with the odor proctor to conduct one or more smell tests at process block 230. For example, the subject, under the instruction provided by the user interface generated on the client, may conduct one or more of a symptom questionnaire, an odor intensity test, odor identification test, and/or odor discrimination test. In some non-limiting examples, the subject may conduct a plurality of odor intensity tests, odor identification tests, and/or odor discrimination tests.

Upon conducting the one or more smell tests, the smell test responses or results from the subject are compiled at process block 240. In one example, this may be achieved automatically using the client 120. That is, after receiving the smell test responses from the subject, the client 120 may store the responses, or forward the responses for storage (e.g., in database 130). In some non-limiting examples, referring to FIGS. 1 and 2, the client 120 may receive input from the subject, and this input may be used to run calculations for the results of the smell tests derived from the algorithms disclosed below. In some configurations, the input received by the client 120 may be used to run calculations via a combination of server(s) 110 and/or client(s) 120. For example, in smell tests administered online or via the app described above, the smell test may be displayed, and the subject's responses may be received via the client 120, but the display may be generated, the subject's responses received, and the calculations to determine the results run on one or more servers 110 at a location remote from the client 120. Server(s) 110 may include a computer or program that provides services to other computers, programs, or users over a computer network, etc.

Finally, at process block 250 a diagnosis may be finalized. In one non-limiting example, a computing device (e.g., client 120 or server(s) 110) may use the responses to assess if the subject has a high risk for having been infected with a target virus or bacteria, such as COVID-19. For example, the computing device may calculate a score based on the responses of the one or more COVID-19 symptom-based questionnaire, odor intensity tests, odor identification tests, and/or odor discrimination tests and, based on that score, identify a subject as having a high risk for being infected with COVID-19.

The above-described method may be further understood by way of specific embodiments of the test guide 152 and odor proctor 150 or test kit. These embodiments are offered for illustrative purposes only, and are not intended to limit the scope of the present disclosure in any way. Indeed, various modifications of the disclosure in addition to those shown and described herein will become apparent to those skilled in the art from the foregoing description and the following embodiments and fall within the scope of the appended claims. Further, the following description may refer to like elements previously described herein. Like elements will be referred to using like reference numerals.

### ODOR PROCTOR

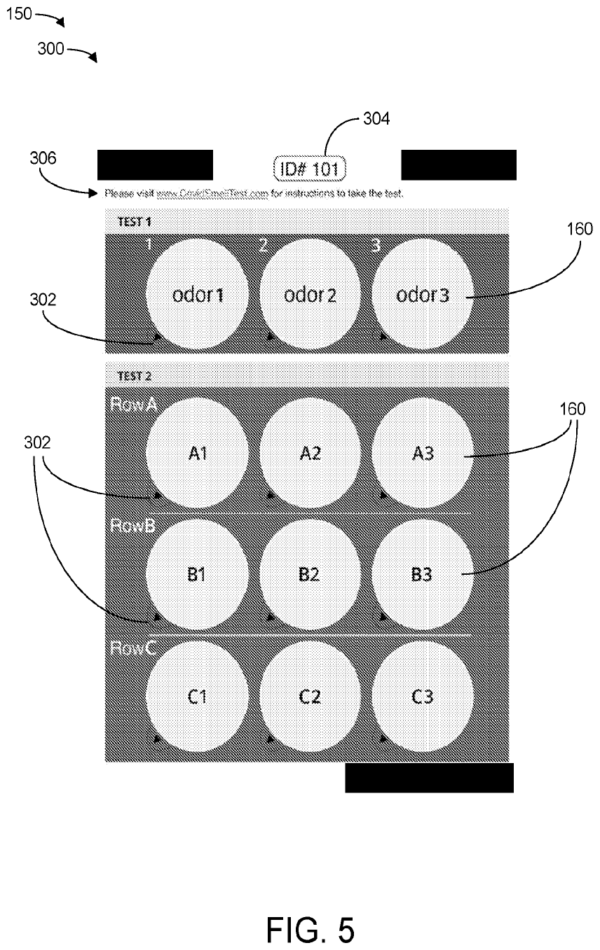

The odor proctor 150 or test kit may be, or include, a printed smell test card. One non-limiting example of a printed test card 300 is illustrated in FIG. 5. The test card 300 can have one or more odors 160 printed thereon. In the illustrated, non-limiting example, the test card 300 can have a plurality or odors 160 printed thereon. The odors 160 on the test card 300 may enable the subject to conduct the one or more smell tests described herein. For example, the smell tests described below may use multiple odors 160. Some non-limiting odors may include menthol, clove, leather, strawberry, lilac, pineapple, smoke, soap, grape, lemon, banana, garlic, cherry, baby powder, grass, fruit punch, peach, chocolate, dirt, popcorn, cola, orange, or any other odor, a combination of which may be delivered to the subject.

Though one example of odor proctor 150 is illustrated, several forms of the odor proctor, each with different odors but the same configuration for longitudinal testing, are utilized. For example, a given person may take a test 1-3 times per week but not smell the same odors or have the same answers for each test. This can prevent subjects from intentionally or inadvertently memorizing the responses or learning the responses from another subject taking the test.

Although 22 specific and non-limiting odors are listed above, the odor proctor 150 may be configured to utilize a wide variety of odors or scents for the administration of the smell tests. For example, the odors may be based on artificial flavorings that are common within the United States (e.g., artificial grape or strawberry flavor). The odors may be based from the actual fruits themselves, as opposed to the artificial flavoring. This may be especially useful for international applications of the present disclosure, where foreign countries may not recognize artificially created odors familiar only to United States residents. Furthermore, the odors for the smell tests may be modified or chosen to be specific to a specific region, culture, gender, age, etc., of the subject.

In the illustrated non-limiting example, the odors 160 on the test card 300 may be individually labeled or stratified into section/rows. For example, the odors 160 may be labeled (e.g., “odor1”, “odor2”, “A1”, “A2”, etc.) such that, when a subject is using the test guide 152, the subject can be instructed on which specific odors 160 to smell. In addition, the odors 160 may be stratified into sections/rows that may correlate to specific smell tests. For example, “Row A”, and odors 160 labeled “A1”, “A2”, and “A3” may be utilized for a specific smell test, as instructed by the test guide 152.

In some of these smell tests, it is important that the subject be able to distinguish between scents. Thus, the test card 300 may be configured to encapsulate each individual odor 160 independently in order to prevent cross-contamination of the scents. For example, the odors 160 may each be encapsulated by a removable cover 302. The cover 302 may be a “peel-and-stick” cover that can be peeled back by the subject prior to smelling the odor 160 and replaced by the subject after smelling the odor 160. Further, this allows the subject to only sample a single odor 160, which can prevent contamination when smelling any adjacent odors or conducting subsequent smell tests.

The test card 300 may also have an identification number 304. The identification number 304 may be unique to each test card 300 such that, when utilizing the test guide, the client 120 may be able to pull the predetermined “correct” responses from storage (e.g., database 130). In this way, the computing device will be able to store or report correct/incorrect responses from the subject and/or identify the specific scent for each odor 160, and the location thereof, printed on the test card 300. For example, the identification number 304 may identify the subject and/or correlate the subject with the unique identification number 304. The subject's data may be correlated to the test card 300 and the smell test may be administered by comparing the subject's answers/responses to the correct odors on the test card 300. According to some non-limiting examples, data and results from the smell tests can be associated with demographics and medical records for the subject. Further, using the identification number 304, the software modules running on server(s) 110 and/or client(s) 120 may generate the display for the subject, including the options presented to the subject, using data stored in database 130 (see FIGS. 1-2) that is correlated to the identification number 304. The software modules may also be configured to store these results in database 130 and link them to the identification number 304.

In the illustrated, non-limiting example, the test card 300 may also have instructions 306 printed thereon. For example, the instructions 306 may instruct the subject on how to access the test guide. In one non-limiting example, the instructions 306 may provide a link to access the test guide (e.g., via a website that can be accessible via the client 120).

The odor proctor 150, in the form of the test card 300, may realize distinct benefits and advantages over current testing methods. For example, utilization of the test card 300 does not require any specialty tools or equipment to conduct the smell test. Thus, the smell tests can be conducted outside of a lab or clinical setting, and further, without the need for a specialist or clinician for the administration of the smell tests. The test card can also be a very cost effective way of administering smell tests, both from a material and logistical standpoint. For example, the test card 300 can be printed (e.g., on paper) and thus be readily manufactured. In addition, when the odor proctor 150 is in a test card 300 format, the test card 300 can be easily delivered to subjects at scale. For example, the test card 300 may be delivered to individuals by mail (e.g., shipped in envelopes). Further, the test card 300 is easily disposable and can be discarded without the need for specialty waste storage equipment.

According to other non-limiting examples, the odor proctor can include an instructional card and an odor kit including a plurality of odor samples or odor delivery devices. For example, an odor kit can include a plurality of spray/mist devices configured to deliver an odor when triggered. According to another example an odor kit can include a plurality of individual containers including an odor in the form of oils. According to yet another example an odor kit can include scented sticks.

### TEST GUIDE

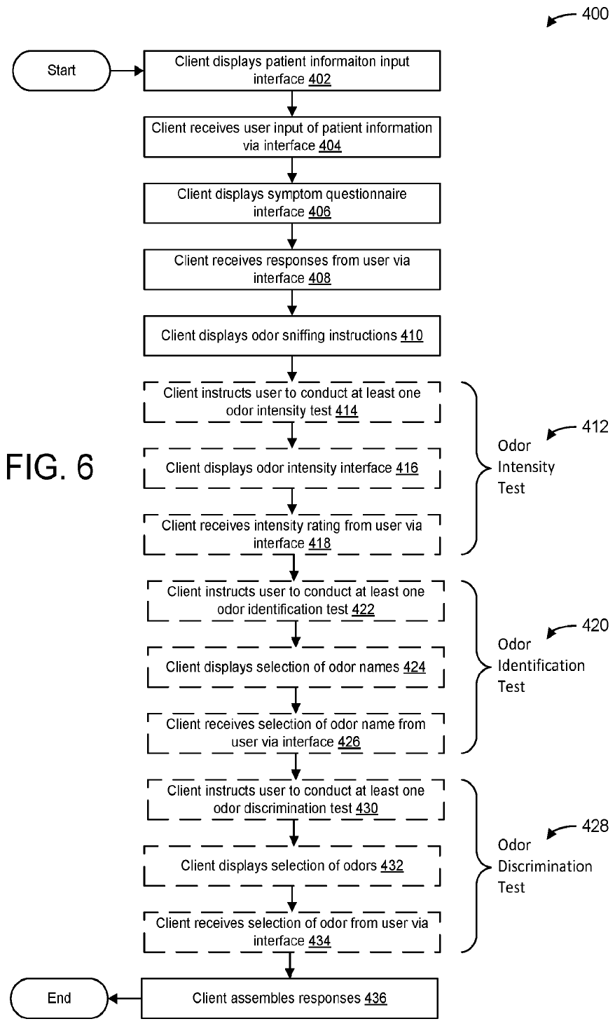

The test kit may further include the test guide 152, which may be used with the odor proctor 150 to perform one or more smell tests (see FIGS. 1-2). As detailed below, the test guide 152 may be realized in software that is readily available via a personal computing device (e.g., client 120, FIG. 1) and configured to generate and display a user interface thereon. FIG. 6 illustrates one such non-limiting example of a method 400 of administering one or more smell tests using the test guide 152 in conjunction with the odor proctor (i.e., utilizing the test kit). The following method will be described with reference to FIGS. 5-13.

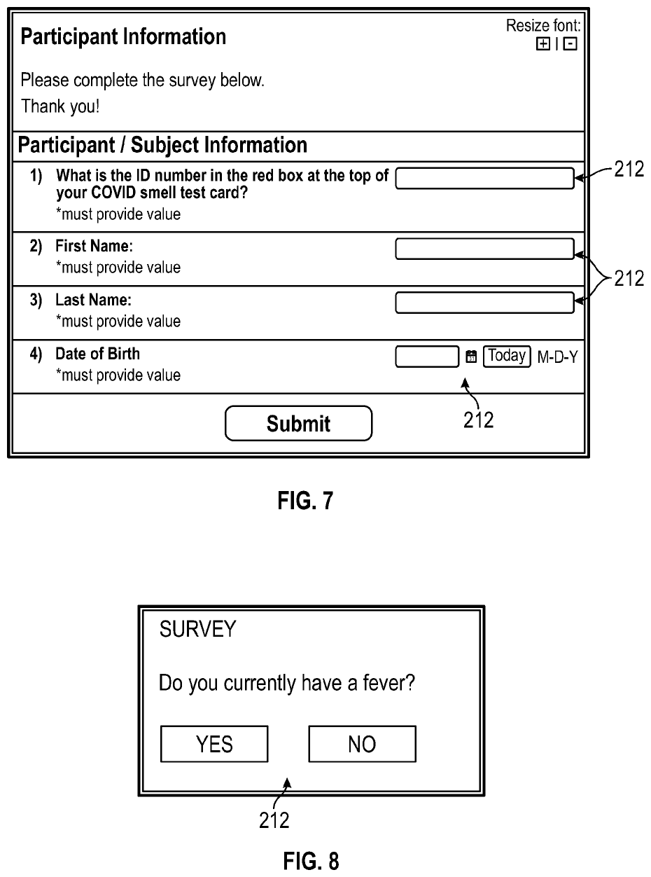

The test guide may begin at process block 402 with the client 120 displaying a patient information input interface. One non-limiting example of a user interface is included in FIG. 7. The patient information input interface may have one or more functions 212 generated thereon. In the illustrated non-limiting example shown in FIG. 7, the patient information input interface may provide a function 212 where the subject can enter the identification number 304 from the test card 300, such as illustrated in FIG. 5. In this way, cards can be generated separately and be distinctly different to present different smells or different arrangements of smells and the system can be paired at the time of testing. According to some non-limiting examples, the identification number can be or include a unique QR code. The patient information input interface may also include functions 212 for the subject to input various other patient information. For example, the subject's name, date of birth, age, sex, ethnicity, and the like. Upon receiving the patient information from the subject at process block 404, the client 120 may correlate the subject, the patient information, and the results of the smell test(s) with the unique identification number 304.

The client 120 may then display a symptom questionnaire interface at process block 406. One non-limiting example is illustrated in FIG. 8. The symptom questionnaire interface can include a function 212 for receiving a subject's response to various questions (e.g., yes/no responses). The questions included in the symptom questionnaire may be specific to a particular disease or pathology (such as COVID-19, as just one example), symptoms, and/or subjective loss of smell/taste. These questions may include, but are not limited to the following: “Do you currently have a fever?”, “Do you currently have a runny nose?”, “Do you currently have a cough?”, “Do you currently have a sore throat?”, “Do you currently have muscle aches?”, “Do you currently have shortness of breath?”, “Are you currently lacking your sense of smell?”, “Is your current sense of smell what it used to be?”, “Is you current sense of taste altered?”, “Are you currently smoking?”, “Do odors cause you to get sick (e.g., asthma)?”, “Have you taken your temperature?”, “What is your current temperature?”, “Have you been skipping meals?”, “Do you currently suffer from smell problems?”, “Do you currently have a ‘cold’ or nasal congestion?”, “Do you have nasal and/or sinus problems (i.e., nasal polyps, chronic sinusitis)?”, “Do you have a history of upper respiratory infection (i.e., viral infection of the nose)?”, “Do you have a respiratory problem such as asthma or emphysema?”, “Are you currently using any inhalers or nasal sprays?”, “Do you have greater difficulty breathing through one nostril versus the other (e.g., a deviated septum)?”, “Have you been tested for COVID-19? If so, what was the result? On what date was the COVID test?” and/or “Are you experiencing unusual fatigue?”. The client 120 may then receive the subject's responses via the functions 212 on the symptom questionnaire interface at process block 408.

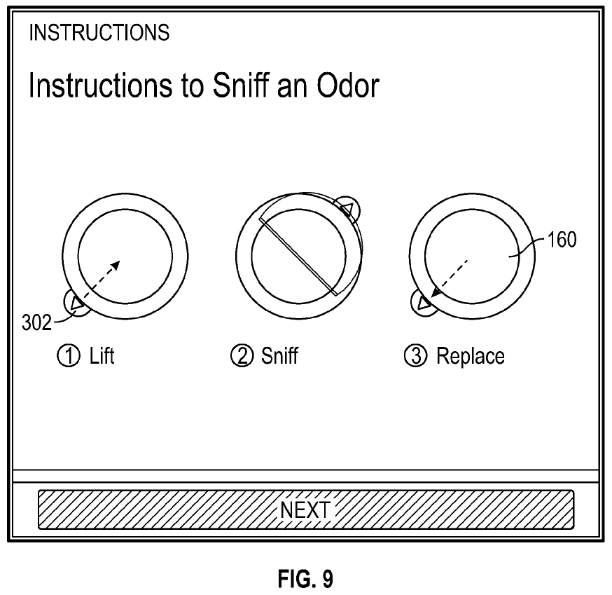

The client 120 may then display odor sniffing instructions to the subject at process block 410. For example, the client 120 may display a screen that provides instructions to a subject on how to conduct a smell test or interact with the test card 300. For example, the client 120 may provide an instructional video, or a series of images containing instructions for carrying out the smell test(s). One such example is provided in FIG. 9. In the illustrated non-limiting example, the client 120 may instruct the subject to at least partially peel back the cover 302 that masks the scent of the odor 160, thereby revealing/delivering the scent to the subject. The client 120 may then instruct the subject to sniff the odor 160 and then replace the cover 302 back onto the odor 160.

After displaying the odor sniffing instructions, the client 120 may then instruct the subject to conduct one or more particular smell tests. That is, as will be described, a variety of different tests can be performed using the test system provided herein. For example, an odor intensity test, an odor identification test, and an odor discrimination test will be described. These are merely examples of tests that can be performed using the systems provided herein. Other tests can be performed. The tests can be performed separately or in different orders. For example, only an odor identification test may be performed. Or, an odor discrimination test may be performed first. The specific type of test used and/or the order they are presented may be selected based a variety of considerations. For example, test types and orders may be varied to keep the subject engaged from day to day in a repetitive or ongoing testing situation. As another example, different tests or orders may be needed for infection diagnosis, as compared to recovery/antibody diagnosis. Thus, the following are just examples and should not be limiting or asserted as “critical,” including the number of tests, order of tests, type of tests, sequencing of tests, repetition of tests, or the like.

In the non-limiting, example, an odor intensity test 412 begins at process block 414. The odor intensity test 412 can provide an objective way of determining olfactory sensitivity. The odor intensity test 412 can include a subject smelling an odor using the odor proctor 150 and providing an objective rating to the client 120 via the user interface to rate the intensity of the scent (e.g., the strength at which the subject detects the odor). In one non-limiting example, the rating may be a numerical scale. The numerical scale may be between zero and ten, where zero may represent an odor that is “not detectable” to the subject and ten may represent an odor that has the “strongest imaginable” scent to the subject.

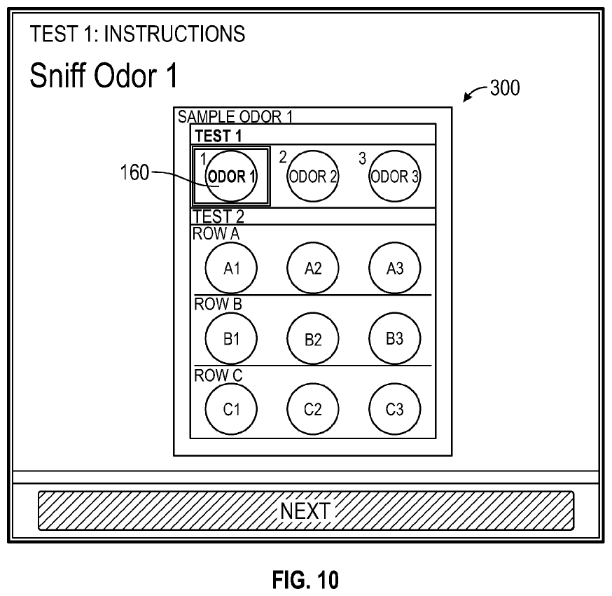

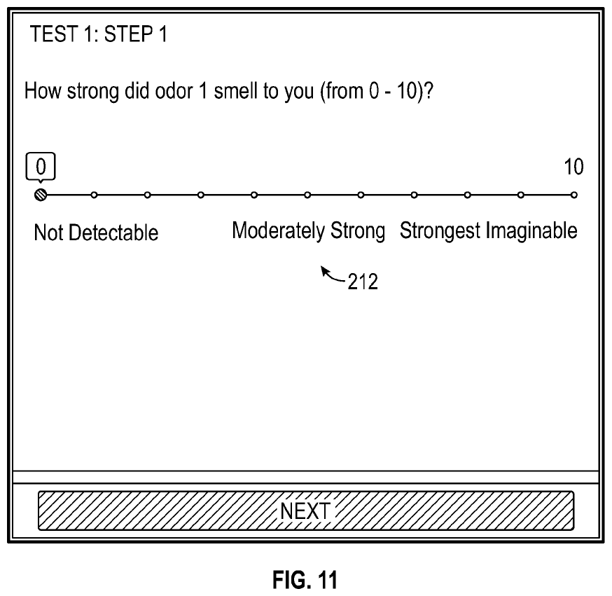

For example, the odor intensity test 412 may begin by the client 120 instructing the subject to smell an odor 160 on the test card 300 at process block 414, such as illustrated in FIG. 10. In the illustrated non-limiting example, the client 120 instructs the subject to smell “odor1” on the test card 300. The client 120 may then display an odor intensity interface at process block 416, such as illustrated in FIG. 11. Upon the subject smelling the odor, the client 120 may then receive an odor intensity input from the subject (e.g., a rating between zero and ten) via a function 212 on the odor intensity interface at 418.

In some non-limiting examples, the client 120 may instruct the subject to conduct a plurality of odor intensity tests 412. For example, the client 120 may repeat process blocks 414 through process block 418 of the method 400 to conduct subsequent odor intensity tests 412. For example, the client 120 may instruct the subject to conduct a first odor intensity test 412 by utilizing the odor 160 labeled “odor1”, a second odor intensity test 412 by utilizing the odor 160 labeled “odor2”, and a third odor intensity test 412 by utilizing the odor 160 labeled “odor3” on the test card 300, such as illustrated in FIG. 5.

The client 120 may instruct the subject to conduct at least one odor identification test 420 at process block 422. The odor identification test 420 can include a subject smelling an odor using the odor proctor 150 and identifying the odor name from a forced-choice list. For example, the forced-choice list can include the names of four odors, where one of the four odor names correctly identifies the odor from the odor proctor 150.

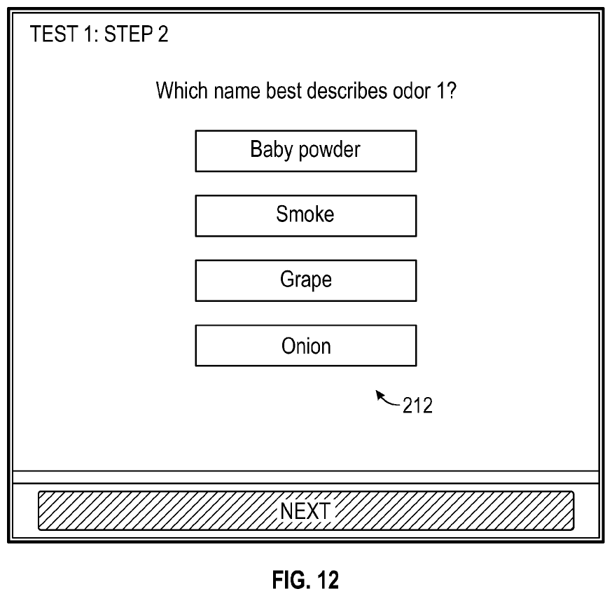

For example, the odor identification test 420 may begin by the client 120 instructing the subject to smell an odor 160 on the test card 300 at process block 422. In one non-limiting example, if previously performed, the odor used in the odor identification test may be the same odor used during the odor intensity test (e.g., “odor1”) on the test card 300. In other non-limiting examples, a different odor may be utilized. In either case, the client 120 may then display an odor identification interface in the form of a selection of odor names at process block 424, such as illustrated in FIG. 12. The client 120 may then receive an odor identification input from the subject (e.g., selecting one of the odors listed) via a function 212 on the odor identification interface at process block 426. In one non-limiting example, the odor identification test 420 can be conducted concurrently with the odor intensity test 412. For example, after the subject enters the odor intensity input during the odor intensity test 412, the client 120 may then display the odor identification interface.

In some non-limiting examples, the client 120 may instruct the subject to conduct a plurality of odor identification tests 420. For example, the client 120 may repeat process block 422 through process block 426 of the method 400 to conduct subsequent odor identification tests 420. For example, the client 120 may instruct the subject to conduct a first odor identification test 420 by utilizing the odor 160 labeled “odor1”, a second odor identification test 420 by utilizing the odor 160 labeled “odor2”, and a third odor identification test 420 by utilizing the odor 160 labeled “odor3” on the test card 300, such as illustrated in FIG. 5.

The client 120 may instruct the subject to conduct at least one odor discrimination test 428 at process block 430. The odor discrimination test 428 can include a subject smelling multiple odors (e.g., three odors) using the odor proctor 150 and identifying the odor that is different from the other odors. For example, the subject may smell three odors 160 on the odor proctor 150, where one of the three odors 160 is different (e.g., two of the three odors are the same scent).

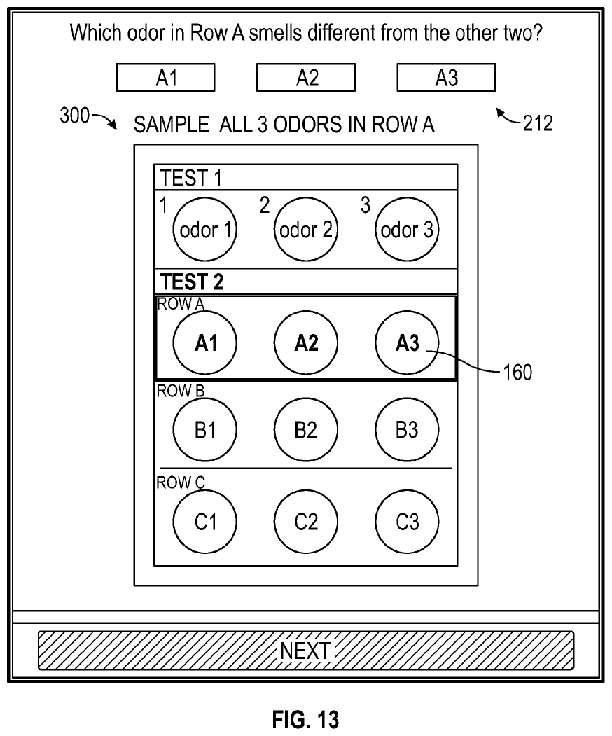

For example, the odor discrimination test 428 (e.g., a triangle odor discrimination test) may begin by the client 120 instructing the subject to smell three odors 160 on the test card 300 at process block 428. In the illustrated non-limiting example, the client 120 instructs the subject to smell odors 160 labeled “A1”, “A2”, and “A3” on the test card 300. The client 120 may then display an odor selection interface at process block 432. One non-limiting example is provided in FIG. 13. Upon the subject smelling the odors, the client 120 may then receive an odor selection input from the subject (e.g., a selection of one of “A1”, “A2”, or “A3”) via a function 212 on the odor selection interface at process block 434.

In some non-limiting examples, the client 120 may instruct the subject to conduct a plurality of odor discrimination tests 428. For example, the client 120 may repeat process block 430 through process block 434 of the method 400 to conduct subsequent odor discrimination tests 428. For example, the client 120 may instruct the subject to conduct a first odor discrimination test 428 by utilizing the odors 160 labeled “A1”, “A2”, and “A3”, a second odor discrimination test 428 by utilizing the odors 160 labeled “B1”, “B2”, and “B3”, and a third odor discrimination test 428 by utilizing the odors 160 labeled “C1”, “C2”, and “C3” on the test card 300, such as illustrated in FIG. 5.

The responses/user inputs received from the subject are then assembled at process block 436. Such response/input assembly may include analyzing and scoring the results, for example, using an algorithm and displaying the results to the user. The responses/input may forwarded to the algorithms disclosed herein for calculating and/or displaying the results of the smell test(s). Further, such assembly at process block 436 may include storing the responses/inputs within the database 130 or on a memory on the client 120, such as illustrated in FIG. 1. For example, the patient information, symptom questionnaire responses, and responses from the odor intensity test(s), odor identification test(s), and odor discrimination test(s) may all be stored within the database 130 or on a memory on the client 120. In one non-limiting example, the responses/user inputs, analysis, and scores of the smell test(s) may be stored as a data record associated with the tested subject.

The test guide disclosed above may realize distinct benefits and advantages over current testing methods. For example, utilization of the test guide does not require any specialty tools or equipment to conduct the smell test. That is, the subject may utilize a personal computer, smart phone, or other mobile device to generate the test guide and conduct the smell tests. Thus, the smell tests can be conducted outside of a lab or clinical setting and instead, for example, may be in the comfort of their own home. Further, by using a subject's personal computing device, a specialist or clinician is not required for the administration of the smell tests. The test guide can also be a very cost effective way of administering smell tests. For example, the majority of subjects already have access to some form of personal computing device and thus the test guide may be readily accessible. That is, the test guide, for example, may be accessed by a website or downloadable via an application on the subject's personal computing device. Further, the multiple smell tests, as described above, may be rapidly administered. In some cases, the smell tests can be conducted in as little as ten minutes, thus preventing the smell tests from occluding the subjects work, lifestyle, etc.

### SMELL TEST RESULT ANALYSIS AND COVID-19 POSITIVE RISK ASSESSMENT

As noted above, a combination of software instructions executed by software modules running on server(s) 110 or client 120 may receive the user inputs from each of the smell tests and may compare the subject's responses against the correct answers to responses to the smell tests received by the user in order to determine if the received answers match the correct answers. The correct answers may be stored in database 130 or on the memory on the client 120 and may be based on, or unique to, each card identification number 304 on the test card 300 (see FIG. 5). The software modules may then run calculations to analyze the subject's answers and generate a score including one or more of the responses from the symptom questionnaire, the number of correct answers for one or more of the odor identification and/or odor discrimination tests, and the intensity ratings for one or more of the odor intensity tests.

The server(s) 110 and/or client(s) 120 may calculate a score for the one or more symptom-based questions from the symptom questionnaire. For example, a score for the one or more symptom-based questions can be based on a binary value, such as a “1” designated for a “YES” response to a question and a “0” designated for a “NO” response, or vise-versa. In that way, a score can be calculated for individual symptom-based question responses, or a cumulative score can be calculated from a plurality of the symptom-based question responses. In this specific non-limiting example, a “YES” response may increase the score. For example, a yes to the symptom question “Have you lost your sense of smell,” can result in an increased score. In another non-limiting example, a score, or portion of a cumulative score, for the symptom-based questions can be based on a numerical value such as the subject's temperature. For example, a higher temperature can result in an increased score.

The server(s) 110 and/or client(s) 120 may calculate a score for the one or more odor intensity tests. In one non-limiting example, a score for the one or more odor intensity tests is based on the numerical rating input by the subject (e.g., between zero and ten). In another non-limiting example, the score for the odor intensity tests may be based on an average of the numerical rating input by the subject. For example, if the subject inputs a higher numerical rating, then the score may increase and if the subject inputs a lower numerical rating, then the score may decrease. The server(s) 110 and/or client(s) 120 may also calculate a score for the one or more odor identification tests. In one non-limiting example, a score for the one or more odor identification tests may be based on the number of correct responses. For example, the score may be based on the number of odors correctly identified by the subject (e.g., 2/3 odors correctly identified, etc.). In another non-limiting example, the score for the odor identification tests may be based on a percentage the number of correct responses. For example, if the subject inputs a correct response, then the score may increase and if the subject does not input a correct response, then the score may decrease. Similarly, the server(s) 110 and/or client(s) 120 may calculate a score for the one or more odor discrimination tests. In one non-limiting example, a score for the one or more odor discrimination tests may be based on the number of correct responses. For example, the score may be based on the number of odor discrimination tests correctly discriminated (e.g., the subject correctly selected the odor that was different from the other odors presented). For example, 2/3 odor discrimination tests correctly discriminated, etc. In another non-limiting example, the score for the odor discrimination tests may be based on a percentage the number of correct responses. For example, if the subject inputs a correct response, then the score may increase and if the subject does not input a correct response, then the score may decrease.

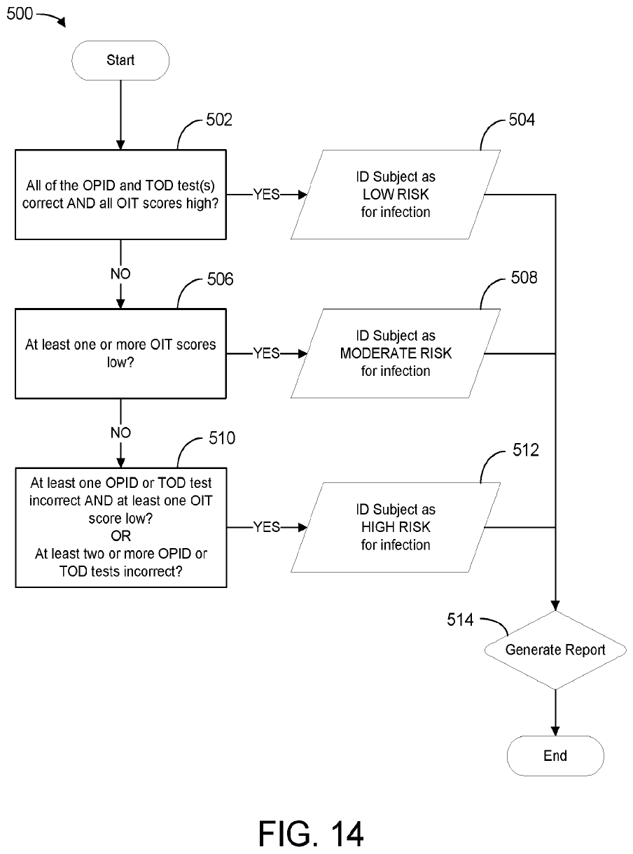

FIG. 14 illustrates one non-limiting example of an algorithm 500 for identifying a subject as having a high risk of being infected with COVID-19. The algorithm 500 is configured to identify a risk level of a subject being infected with COVID-19 based on one or more of the smell test scores previously described herein. For example, the algorithm 500 can be executed by the server(s) 110 and/or client(s) 120 to determine a risk level of the subject being infected with COVID-19. The determination of the risk level of infection may be based on at least one or more of the scores from the symptom questionnaire responses, odor intensity test(s) (“OIT”), odor percept identification test(s) (“OPID”), triangle odor discrimination test(s) (“TOD”), and/or a combination thereof.

In some aspects of the algorithm 500, the server 110 or client 120 may need to determine if the subject had a “high” or “low” odor identification or odor discrimination test score. In one non-limiting example, the odor identification/discrimination test scores may be compared to a predetermined threshold. For example, the predetermined threshold may be based on the number of correct responses to the odor identification/discrimination tests (e.g., 1/3 correct, 2/3 correct, 3/3 correct, etc.). In one exemplary non-limiting example, the predetermined threshold may be defined at 2/3 odor identification/discrimination tests, in which case if the subject correctly responded to 1/3 odor identification/discrimination tests, the score would be below the predetermined threshold. It is to be understood that percentages could also be used and the predetermined threshold could be a percentage value indicative of a desired score for the odor identification/discrimination tests (e.g., 33%, 66%, etc.)

In some aspects of the algorithm 500, the server 110 or client 120 may need to determine if the subject had a “high” or “low” odor intensity test score. In one non-limiting example, the odor intensity test scores may be compared to a predetermined threshold. For example, if the odor intensity test score is above the predetermined threshold, the score may be regarded as “high.” Similarly, if the odor intensity test score is below the predetermined threshold, the score may be regarded as “low.” As one non-limiting example, if a user is prompted with an intensity scale ranging from 0-10, a threshold of 6 or less may be used for conferring risk, where 7 or higher is considered normal. As another non-limiting example, an average intensity score or the intensity score on “odor 1” may serve as a threshold. For example, only one of the odor intensity scores may be used, or an average of three scores for three odor intensity tests may be used.

In the illustrated non-limiting example, the algorithm 500 may begin at process block 502 where the server 110 or client 120 may determine if the subject correctly responded to each of the administered odor identification tests and odor discrimination tests and if the subject had a high odor intensity test score relative to a predetermined threshold. If the server 110 or client 120 determines that the subject correctly responded to all of the odor identification tests and odor discrimination tests and if the subject had a high odor intensity test score (i.e., based on the responses stored within the database 130 or on a memory on the client 120, see FIG. 1), then the client 120 or server 110 may identify the subject as having a “low risk” for being infected with COVID-19 at process block 504.

If the server 110 or client 120 determines that the subject did not correctly respond to each of the administered odor identification tests and odor discrimination tests and/or if the subject did not have a high odor intensity test score, then the server 110 or client 120 may proceed to process block 506 where the server 110 or client 120 may determine if the subject had a low odor intensity test score on at least one or more odor intensity tests relative to a predetermined threshold. If the server 110 or client 120 determines that the subject had a low odor intensity test score on at least one or more odor intensity tests, then the client 120 or server 110 may identify the subject as having a “moderate risk” for being infected with COVID-19 at process block 508.

If the server 110 or client 120 determines that the subject did not have a low odor intensity test score on at least one or more odor intensity tests, then the server 110 or client 120 may proceed to process block 510 where the server 110 or client 120 may determine if the subject incorrectly responded to at least one odor percept identification or one odor discrimination test and had at least one low score on an odor intensity test. If the server 110 or client 120 determines that the subject incorrectly responded to at least one of the administered odor percept identification or one odor discrimination test and had at least one low score on an odor intensity test then the client 120 or server 110 may identify the subject as having a “high risk” for being infected with COVID-19 at process block 512. Alternatively, at process block 510 the server 110 or client 120 may determine if the subject incorrectly responded to at least two of the administered odor percept identification or odor discrimination tests. If the server 110 or client 120 determines that the subject incorrectly responded to at least two odor percept identification or odor discrimination tests (or one of each) then the client 120 or server 110 may identify the subject as having a “high risk” for being infected with COVID-19 at process block 512.

Upon the determination of a risk level of the subject being infected with COVID-19 (e.g., low, moderate, high), the client 120 may store the risk level within the database 130 or on a memory on the client 120, possibly as one or more data records associated with the subject. The server 110 or client 120 may then generate a report of the risk level assessment at process block 514. The generated report, including the results of the smell tests and interpretation of the results, may then be displayed to the subject via the client 120.

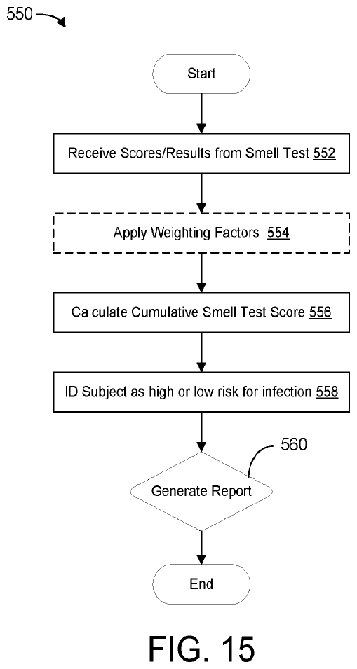

FIG. 15 illustrates one non-limiting example of an algorithm 550 for identifying a subject as having a high risk of being infected with COVID-19. The algorithm 550 is configured to identify a risk level of a subject being infected with COVID-19 based on one or more of the smell test scores previously described herein. For example, the algorithm 550 can be executed by the server(s) 110 and/or client(s) 120 to determine a risk level of the subject being infected with COVID-19.

The determination of the risk level of infection may be based on at least one or more of the scores from the symptom questionnaire responses, odor intensity test(s) (“OIT”), odor percept identification test(s) (“OPID”), triangle odor discrimination test(s) (“TOD”), and/or a combination thereof. Combinations can include variables such as a subject incorrectly responding to one or more (e.g., two or more, etc.) of the odor identification/discrimination tests. Other combinations can include engineered variables such as a subject incorrectly responding to one or more (e.g., two or more, etc.) of the odor identification/discrimination tests and providing a low score (e.g., relative to a predetermined threshold) to one or more odor intensity tests. It is to be understood by one of ordinary skill in the art that various other combinations can also be included to develop engineered variables, including combinations of one or more responses from the symptom questionnaire.

The algorithm 550 can begin at process block 552 where the server 110 or client 120 can receive the calculated scores/results from the smell test including scores from the symptom questionnaire responses (e.g., 0 or 1 correlating to a “no” or “yes” response, respectively), odor intensity test(s) (e.g., a rating between 1 and 10), odor percept identification test(s) (e.g., a 0 or 1 for a correct or incorrect response, respectively, or a number of correct or percentage correct), triangle odor discrimination test(s) (e.g., a 0 or 1 for a correct or incorrect response, respectively, or a number of correct or percentage correct), and engineered variables such as those described above. The scores for the engineered variables can be binary such as a 1 if the subject met the variable criteria (such as missing two or more of the OPID or TOD tests) and a 0 if the subject did not meet the criteria, or an average score based on the scores for the tests included in the engineering variable.

Next, at process block 554, the server 110 or client 120 can apply weighting factors to one or more of the scores from the smell test including scores from the symptom questionnaire responses, odor percept identification test(s), triangle odor discrimination test(s), and engineered variables. For example, the score from a first symptom questionnaire response can have a first weighting factor, the score from a first odor intensity test can have a second weighting factor, the score from the first odor identification test can have a third weighting factor, and so on. That is, each of the scores from individual responses from the symptom questionnaire and the scores from each of the odor intensity, identification, and discrimination tests can have their own unique weighting factor. According to some non-limiting examples, the weighting factors can be based on a plurality of previously entered performance data stored in the database 130 corresponding to results from previously conducted smell tests.

According to one non-limiting example, the server 110 or client 120 can apply logistic regression analysis to the plurality of previously entered performance data and derive weighting factors or coefficients based on the previously entered performance data. These unique weighting factors are then used as a scalar for their respective test score or engineered variable. For example, the score for the odor intensity test can be multiplied by a weighting factor unique to that odor intensity test. According to some non-limiting examples, the weighting factors can be between 0 and 1.

Next, at process block 556, the server 110 or client 120 can calculate a cumulative smell test score based on one or more of the individual scores from the smell test, after being weighted by their respective weighting factors, including scores from the symptom questionnaire responses, odor percept identification test(s), triangle odor discrimination test(s), and engineered variables. According to one non-limiting example, the cumulative score is summation of the weighted scores from one or more of the symptom questionnaire responses, odor percept identification test(s), triangle odor discrimination test(s), and engineered variables.

Next, at process block 558, the server 110 or client 120 can determine if a subject is at high risk (e.g., smell altered) or low risk (e.g., smell normal) for having COVID-19 infection. For example, the cumulative score from the smell test can be compared to a predetermined threshold, where if the cumulative score is above the predetermined threshold, the subject is identified as having a high-risk for being infected with COVID-19, and if the cumulative score is below the predetermined threshold, the subject is identified as having a low-risk for being infected with COVID-19. According to some non-limiting examples, the predetermined threshold can be based on the plurality of previously entered performance data from previous studies or test results correlating the smell test scores of verified infected/non-infected subjects. According to some non-limiting examples, the predetermined threshold can be between 0 and 1.

Upon the determination of a risk level of the subject being infected with COVID-19 (e.g., low or high), the client 120 may store the risk level within the database 130 or on a memory on the client 120, possibly as one or more data records associated with the subject. The server 110 or client 120 may then generate a report of the risk level assessment at process block 560. The generated report, including the results of the smell tests and interpretation of the results, may then be displayed to the subject via the client 120.

Various modifications of the systems, methods, and algorithms described herein are also envisioned. For example, the predetermined threshold for the odor intensity test score (e.g., the threshold for the determination of a “high” or “low” score) may be relative or dependent on various factors. In one non-limiting example, the database 130 may include previously-entered performance data (e.g., studies, test results, historical data) relevant to odor intensity, identification, and discrimination test results. For example, the previously-entered performance data can include data from previous studies and/or entered by a data or system administrator. The previously-entered performance data can also include smell test results from various subjects who have previously taken the smell test using the test guide and odor proctor described herein and those smell test results can be linked to a verified COVID-19 test to correlate the smell test results with verified COVID-19 positive or negative infected patients.

In one non-limiting example, upon the subject entering the patient information into the client 120, the server 110 or client 120 may query the database 130 for the previously-entered performance data. Using this previously-entered performance data returned from the database 130 query, the software modules on the server 110 or client 120 may calculate a threshold for the odor intensity test score that can be unique to the subject. For example, the threshold may be determined based on age, gender, culture, ethnicity, country, region, and/or other various factors that may influence olfactory sensitivity.

In another non-limiting example, machine learning processes may be implemented by the client 120 and/or the server 110 to adapt aspects of the algorithm described herein. For example, the client 120 and/or the server 110 may use machine learning techniques to adjust the score thresholds previously described herein as more data is gathered in the database 130. Additionally, the client 120 and/or the server 110 may use machine learning techniques to adjust the thresholds/score requirements at which a user is identified as having a risk for infection based on the aggregate of data (e.g., previously-entered performance data, etc.) stored in the database 130.

### EARLY ASYMPTOMATIC DETECTION

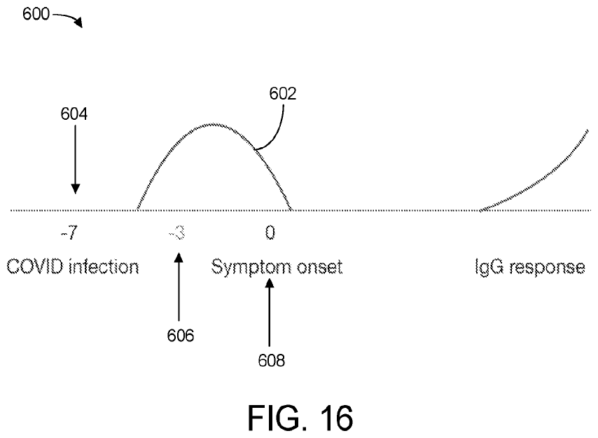

Absent the present disclosure, there are no current testing methods capable of detecting or diagnosing the early asymptomatic stage of the coronavirus infection. As detailed below, the present systems, methods, and algorithms described herein may detect the early stage of the coronavirus or COVID-19 infection. COVID-19 may uniquely manifest itself by way of loss of smell or olfactory function. For example, FIG. 16 illustrates a life cycle 600 of COVID-19 viral load 602. As illustrated, a subject may be infected for about seven to ten days (at 604) or more before being symptomatic (e.g., fever, etc.). However, the loss of smell or olfactory function can occur about three or more days prior to being symptomatic (at 606). Further, the loss of olfactory function may occur at a time when the viral load 602 is high, especially when compared to the viral load at symptom onset (at 608). Due to the high viral load 602 at the onset of the loss of olfactory function, the testing and detection of the PCR and antigen tests thereof may be more accurate. Thus, the detection of olfactory deficiencies, such as a lower threshold for smell intensity recognition, odor identification, or odor discrimination may be a biomarker for the early onset of COVID-19.

### CLINICAL DECISION MAKING

The smell test results disclosed herein may also be utilized in a clinical environment to aid in the treatment or diagnosis of COVID-19. For example, if the algorithms disclosed herein identify a subject as having a high risk of being infected with COVID-19, the client 120 may alert the subject to get tested using the nasopharyngeal swab SARS-COV-2 RT-PCR test or other form of COVID-19 diagnostic test to confirm a diagnosis of being infected with SARS-COV-2. That is, the small test(s) can be used to assess olfactory loss related to SARS-COV-2 infection and then a subject can be instructed to take a diagnostic test to confirm the infection. The integration of the NP RT-PCR test and the smell tests disclosed herein may further the accuracy of COVID-19 testing and, more importantly, can identify subjects early on of the potential for infection, which may lead to a more successful treatment of the subject and help the subject in determining self-quarantine measures. For example, the smell test methods described herein can be utilized to increase pre-test probability of COVID-19 testing (such as NP RT-PCR testing). The smell tests systems and methods disclosed herein may also be used in conjunction with a serology test for SARS-COV-2, an antigen test for SARS-COV-2-19, and other tests to demonstrate infection with SARS-COV-2.

The smell test results may also be used to monitor patients for the probability of subsequent symptoms or illnesses. That is, the smell test may be used as an orthogonal test with patients who are known to be infected to determine if they are at risk of respiratory compromise. For example, the loss of olfactory function may be an indicator that a subject may have a low probability of developing pneumonia. Additionally, the smell tests may be used to help rule out COVID-19 infection in patients who are ready to be discharged from the hospital. As such, the tests results can be a useful tool for a clinician in determining treatment protocols for a particular subject, which may provide a more efficient and informed clinical environment.

### POST-VIRUS DIAGNOSIS AND RECOVERY

The smell test results and the data collections thereof may also provide a useful tool in recovery tracking and post COVID-19 diagnosis recovery monitoring. For example, the tests may be configured such that a subject takes numerous consecutive smell tests over a period of time (e.g., once a day, once a week, once a month, etc.). The results from the consecutive smell tests may be stored (e.g., in the database 130) and can be used for the identification of the subject's recovery or the identification of a subjects period of being contagious. That is, the results may be stored and tracked to determine if a subject has either recovered or is identified as no longer being contagious. This may provide a way to enable a determination that a subject may, for example, return to work or from school, or come out of quarantine.

In some non-limiting examples, this tracking may be conducted by the monitoring of smell test scores from one or more of the odor intensity tests, odor identification tests, and/or odor discrimination tests. For example, a recovery of the subject may be defined by a threshold of improvement in the smell test scores (e.g., an improvement of a score by 10%, 20%, 30%, etc.). In other non-limiting examples, a recovery may be defined by the scores of the odor intensity tests becoming above the predetermined threshold previously described herein.

The integration of antibody or blood testing and the smell tests disclosed herein may also further the accuracy of identifying a subject as being recovered from COVID-19. For example, if the algorithms disclosed herein identify a subject as being recovered from COVID-19, the client 120 may alert the subject to get tested using the blood or antibody tests.

The above-described systems and methods may be further understood by way of additional examples. These examples are offered for illustrative purposes only, and are not intended to limit the scope of the present disclosure in any way. Indeed, various modifications of the disclosure in addition to those shown and described herein will become apparent to those skilled in the art from the foregoing description and the following examples and fall within the scope of the appended claims.

### EXAMPLE 1

In one non-limiting example showcasing the efficacy of the methods described herein, a research study was conducted relating the ability to identify olfactory deficits to biomarkers of COVID-19 infection. The example study explained below should in no way limit the scope of the present disclosure.

Post-infectious-anosmias typically follow death of olfactory sensory neurons (OSNs) with a months-long recovery phase associated with parosmias. While profound anosmia is the leading symptom associated with COVID-19 infection, many patients regain olfactory function within days to weeks without distortions. For example, RNA levels of odorant receptors are markedly reduced in OSNs in a non-cell autonomous manner. Further, subjects infected with COVID-19 may rate odors with lower intensities. In some cases, subjects infected with COVID-19 may have odor discrimination deficits relative to people that tested negative for COVID-19. The inflammatory-mediated loss of odorant receptor expression with preserved circuit integrity can account for the profound anosmia and rapid recovery of olfactory function without parosmias caused by COVID-19.

Subjective reduction of smell (hyposmia) commonly occurs during upper respiratory viral infections (URIs), and resolves concomitantly with improvement in rhinorrhea and nasal congestion symptoms. About 5% of patients experience a post-infectious, prolonged olfactory disorder that often recovers over 6-12 months with odor training. By contrast, a much larger proportion of patients infected with the SARS-COV-2 virus (e.g., 34-65%) self-report anosmia, usually without accompanying rhinorrhea or nasal congestion. However, self-report of smell loss is often unreliable. Further, in many cases, olfactory deficits occur before the onset of other symptoms of a COVID-19 disease or are the only manifestation of the disease.

Viral induced rhinorrhea, or “runny nose” is one mechanism that may contribute to olfactory dysfunction by preventing odorants from reaching odorant receptors (“OR”). Infection with SARS-COV-2, however, is not commonly associated with rhinorrhea or congestion. Viral killing of olfactory sensory neurons (“OSNs”), is another mechanism of olfactory dysfunction, and regeneration of the OSNs from stem cells and reintegration of newly differentiated neurons into existing circuits can be responsible for the months-long recovery process. The rate of recovery of olfactory function is another distinguishing feature of COVID-19 associated anosmia relative to other post-infectious olfactory deficits. In a recent longitudinal survey, 80% of patients reported subjective partial or full recovery after 1 week, rather than months as typically described by post-viral smell loss patients. Together, these distinguishing clinical features (i.e., lack of rhinorrhea or congestion, the broad penetrance of hyposmia, and the rapid recovery of olfactory function) show that COVID-19 infection can induce olfactory loss via a mechanism that is distinct from a neurotoxic effect mediated by other viruses.

In an initial test, olfactory function was characterized in non-hospitalized patients that tested positive or negative for the COVID-19 infection by nasopharyngeal RT-PCR for SARS-COV-2. The non-hospitalized patients with COVID-19 infection may score intensities of odors significantly lower and discriminate between odors with less acuity relative to patients with negative COVID-19 testing. These phenotypes may be consistent with a peripheral mechanism of olfactory dysfunction, and consistent with loss of OR expression. Profound reduction of OR expression in the setting of robust anti-viral innate immune signaling in the olfactory epithelium may resolve the paradox of why SARS-COV-2 robustly affects smell function even though OSNs may not express the primary entry receptors for the virus; this mechanism of OSN dysfunction without death may also explain why the recovery of profound olfactory function occurs on a short time scale relative to other post-infectious anosmias.

COVID-19 infection of respiratory epithelial and sustentacular cells can lead to local innate immune activation, even in the absence of constitutional symptoms to reduce OR expression in OSNs. To test this in humans, an olfactory battery can be integrated into a smell test, such as the systems and methods described herein, for probing olfactory function in people with a possible or definite exposure to SARS-COV-2 virus to identify a risk level for determining if the people have SARS-COV-2. The COVID smell test battery used in this study consisted of three sequential tests: 1) rate the intensity of each odor (e.g., an odor intensity test); 2) odor percept identification (e.g., an odor identification test); and 3) odor discrimination (e.g., an odor discrimination test). The odors were presented on an odor proctor as “peel and sniff” labels arrayed on a card (e.g., the test card 300, see FIG. 5) and recorded responses on a web-based app (e.g., the test guide disclosed herein, see FIGS. 7-13).

Patients with documented COVID-19 by nasopharyngeal swab RT-PCR tests (n=18) and patients with one or more negative SARS-COV-2 RT PCR test results (n=16) were used to take the COVID smell test battery. The distribution of age, sex, time between the RT-PCR assay and smell test, and subjective complaints of smell loss were not statistically different between the two cohorts (see Table 1).

TABLE 1

| | COVID+ | COVID− | |
|---|---|---|---|
| Demographics | Patients (n = 18) | Patients (n = 16) | p-value |
| Mean Age, yrs (SD) | 35.8 (3.4) | 37.7 (4.2) | 0.74^{a} |
| % Female (N) | 70% | 64% | 0.72^{b} |
| % Reporting Smell Loss | 50% | 19% | 0.12^{b} |
| at Time of Smell Test | | | |
| Time Between COVID | 4.4 (3.0) | 12.8 (3.4) | 0.1^{a} |
| test and Smell Test | | | |

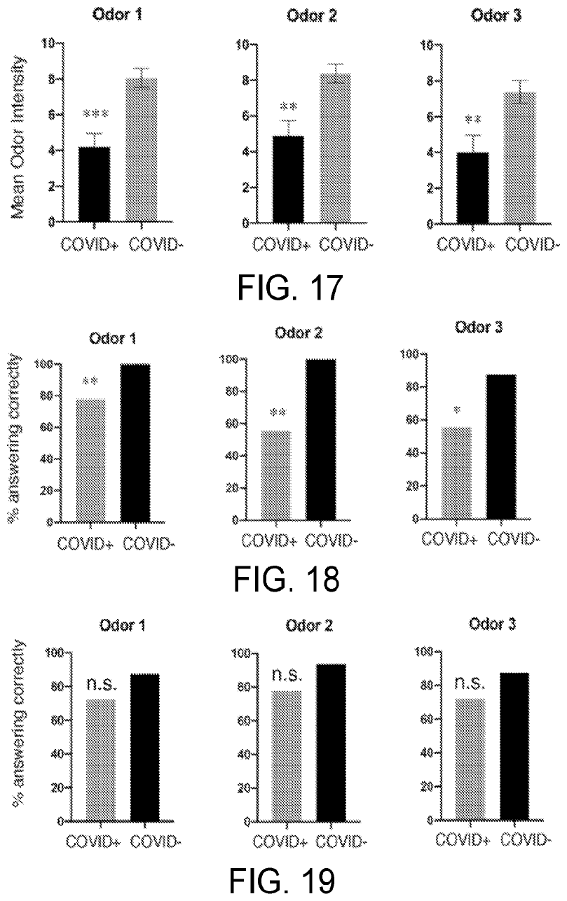

FIG. 17 illustrates that COVID-19 patients demonstrated reduced subjective intensity ratings for all three odors compared to participants with negative SARS-COV-2 test results. FIG. 18 illustrates that COVID-19 patients demonstrated diminished performance on the odor discrimination test compared to participants with negative SARS-COV-2 test results. FIG. 19 illustrates that COVID-19 patients demonstrated reduced performance on the 3-item odor identification test compared to participants with negative SARS-COV-2 test results. Together, the reduced intensities and difficulty with identifying odors or discriminating three different presented odors is consistent with a peripheral mechanism of olfactory dysfunction. Further, using the algorithms disclosed herein (e.g., algorithms 500, 550, FIGS. 14 and 15), the smell test resulted in 94% sensitivity of positively identifying COVID-19 infected subjects, far outweighing the accuracy of other current testing methods used in the art.

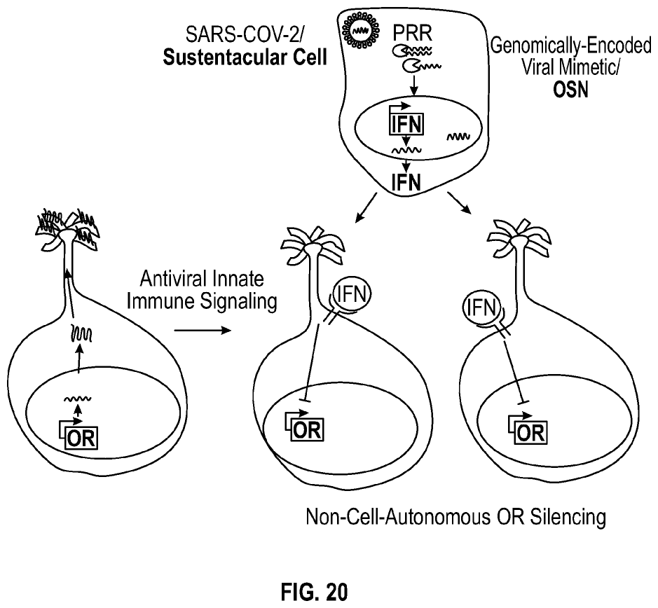

FIG. 20 illustrates a model for non-cell-autonomous innate immune inhibition of odorant receptor expression. All cells in the olfactory epithelium, including mature OSNs express pattern-recognition receptors (PRR) that can detect cytoplasmic or endosomal nucleic acids. For example, the positive RNA single stranded genome of Coronaviruses, or its double-stranded RNA intermediates, can be detected by PRRs. PRRs then activate the transcription of interferon (IFNs) cytokines, and other cytokines and chemokines, which are secreted from infected cells. IFNs then trigger the activation of a host of genes that define a type I interferon antiviral innate immune response. During COVID-19, even though OSNs do not express the main entry receptors for SARS-COV-2, they can still detect IFNs, other cytokines and chemokines, from sustentacular or other neighboring cells, which activate a robust IFN-I response in OSNs, leading to repression of OR transcription. Olfactory deficits in the setting of COVID-19 infection may operate via an innate immune, e.g., IFN-I induced mechanism. A hyperactive immune response, including cytokines and chemokines, triggered by SARS-COV-2 may mediate the respiratory failure in patients with COVID-19. Moreover, SARS-COV, the closest relative of SARS-COV-2 with 79% sequence homology, can trigger a toxic IFN-I antiviral innate immune response that causes deadly respiratory failure in humans.

In general, inflammatory-mediated loss of odorant receptor expression with preserved circuit integrity can account for the profound anosmia and rapid recovery of olfactory function without parosmias caused by COVID-19. Further, this study illustrates that the self-administered, objective smell testing described herein may identify a biomarker for the inflammatory status in the olfactory epithelium evoked COVID-19.

### EXAMPLE 2

In another non-limiting example showcasing the efficacy of the methods described herein, a clinical study was conducted utilizing the test proctor in the form of a printed test card in combination with the test guide in the form of a web-based app, such as the test card 300 of FIG. 5 and the test guide illustrated in FIGS. 7-13. The example study explained below should in no way limit the scope of the present disclosure. The smell test(s) described herein and utilized in this clinical study were developed to detect smell loss related to COVID-19 infection, as opposed to other causes of smell loss, such as Parkinson's disease, Alzheimer's disease, and traumatic brain injury, based on a unique mechanism of smell loss based on sterile inflammation in the primary olfactory sensory neurons since receptors for the virus are not expressed on the surface of primary olfactory neurons. Sterile inflammation causes a profound reduction in the expression of odorant receptors in the olfactory sensory neurons in the mouse olfactory neuroepithelium. Loss of receptor density can impact intensity ratings of odors and impair discrimination of three rapidly presented strong odors due to rapid habituation. This alterations in the olfactory neural circuit are distinct from the brain pathology seen in Parkinson's disease, Alzheimer's disease, and traumatic brain injury as well as the axon shearing of primary olfactory neurons that can occur in traumatic brain injury.

The primary objective of the study was to implement a new web-based application paired with a smell card, the COVID Smell Test, to collect symptoms and to administer olfactory tests to quantify smell function in subjects with documented COVID-19 positive and/or COVID-19 negative infection by RT-PCR assay and to study the utility of olfactory deficits in identifying patients with COVID-19 (SARS-COV-2) infection.

The app-based olfactory test battery was adapted to afford objective smell, testing patients with a possible or definite exposure to COVID-19. Many patients infected with COVID-19 experience a partial or complete loss of their sense of smell. Objective testing of smell function will validate smell loss in COVID-19 positive patients and afford the opportunity to follow their smell function longitudinally to chart the kinetics of recovery. With smell loss validated as a sign of COVID-19 in individuals with and without symptoms and those with generic symptoms (cough, fever, sore throat, etc.), then the COVID Smell Test may be used as an adjunct screening tool administered at the time of COVID-19 testing, with results and interpretation provided immediately. To reduce the risk of transmissibility afforded by the COVID Smell Test, the test employs a disposable odor card, a simplified 3-odor identification test (including odor intensity tests) and a 3-trial odor discrimination test.

In the clinical study the following was addressed: (1) odor discrimination and odor identification scores in COVID-19 positive patients are be reduced relative to historic controls (bona fide COVID-19 negative patients) using the same odors and in COVID-19 negative patients; and (2) reduced odor discrimination and/or reduced odor identification scores increase pre-test probability of COVID-19-positive testing.

Subjects used their uniquely numbered COVID Smell Test card given to them. The smell card directed them to the web-based app (e.g., opening a browser and going to a website on their compatible smartphone, tablet, or computer). After entering their COVID Smell Test card number, the app seamlessly moved the subject's webpage to a patient info information screen, where the subject entered their name, their birthdate, their mailing address, and their unique smell card number. Then the next window automatically took them to a symptom questionnaire screen where subjects answered questions from a symptom tracking survey. Next, subjects self-administered the COVID Smell Test by peeling labels on the smell card and smelling the odors as directed by the web-based app. Lastly, subjects entered responses to question and prompts that comprised of the tests for odor intensity rating, odor percept identification (i.e., Test 1) and odor discrimination (i.e., Test 2).

The results from the questionnaire and the tests were combined with the unique ID# on the COVID Smell Test card for analysis. The unique ID# on the COVID Smell Test card was entered by the subject into the app at the start of olfactory testing, allowing physicians to tie olfactory results with patient medical information for data analysis including COVID-19 status and other variables related to the course of the infection and treatment.

It was anticipated that the following sequence of study procedures would take less than 15 minutes to conduct. First, the Symptom Tracking Survey. Second, Test 1 (3 trials, 1 odor per trial) including an intensity test and an identification test. In the intensity test subjects were instructed by the web-based app to peel the odor label for each trial and smell the odor. Subjects were asked to rate the intensity of the odor using a visual analog scale from 0-10 with the anchoring terms of no odor perception for zero and strongest odor imaginable for 10. In the identification tests subjects were then asked to identify the odor through a 4-option forced-choice list of odor names as cues. The inputs taken from the subject by the web-based app were: (a) a measure the intensity rating for each odor label sniffed; and (b) a measure the number of correct odor percept identifications. Next the subjects performed Test 2 (3 trials, 3 odors per trial). In the discrimination test, subjects were instructed by the app to peel and smell a panel of three odor labels for each trial. Subjects were asked to identify which of the three odors smells different from the other two. This was completed three times with Row A, Row B, and Row C (see, e.g., FIG. 5). The inputs taken from the subject by the web-based app were a measure the number of correct discrimination trials.

The odor identification and discrimination question responses with binary responses (correct, incorrect), the numeric rating measures of odor intensity, the reported symptom variables (presence/absence of fever, lost sense of smell, taste, etc.), and various derived measures thereof, were evaluated for association with RT-PCR outcome of presence/absence of the SARS-COV-2 (COVID-19) virus at any point (CovPosEver). First, univariate tests relating each smell/symptom predictor to RT-PCR outcome were conducted. For binary or categorical predictors, the chi-square test of association, or Fishers Exact Test (if 25% or more cells had expected frequencies <5) was used. For numeric predictors, distributions could generally not be assumed normal, so the nonparametric Mann-Whitney test was employed.

These univariate tests answer the question of whether each given analyzed predictor significantly predicts RT-PCR outcome in the absence of any other information (or adjustment for covariates). A multivariable assessment with a number of pertinent predictors (and covariates), was used to model whether certain predictors are the underlying dominant predictors that provide significantly increased predictability beyond what other predictors provide even when adjusted for them (and covariates). The multivariable model also produces the optimal linear combination of predictors that best predicts the dependent variable (positive or negative RT-PCR result). Multiple logistic regression with binary RT-PCR result as the dependent variable and a set of eleven predictors/covariate terms was used for the multivariable assessment. The eleven predictors/covariate terms were chosen because they were significant predictors in univariate testing and/or they were substantively relevant predictor or covariate terms.

A backward elimination procedure was employed to reduce the number of predictors. This method makes the decision to eliminate a predictor based on adjustment for all other potentially effective predictors (and confounds) still in the model, disregarding only predictors already proven non-significant at that point. The predictors included in the initial model were the number of wrong answers of the six-odor identification and discrimination questions, reported loss of sense of smell, not normal sense of smell, altered taste, and average correct of the three odor identification questions (where each is coded 0 or 1 for incorrect, correct answers respectively), average intensity of the three odors, and average discrimination correct. Covariates were sex, age (linear and quadratic terms), the interaction of age X average identity, and the ordinal day of the study from the first smell date. The latter was employed because the likelihood of RT-PCR outcome of presence/absence of the SARS-COV-2 virus (CovPosEver=Yes) was known from preliminary statistics and graphical analysis to decline as the study progressed, presumably due to the COVID-19 disease expending itself. Significance tests for the final model as a whole, as well as for individual predictors were produced as well as odds ratio estimates with confidence intervals, and area under the ROC curve.

Data entered into the app interface was available to physicians through a web-based secured dashboard, that enables physicians to view and export summary data for each subject in real-time.

For the COVID Smell Test, associations between COVID-19 positive and COVID-19 negative patients were be measured generalized linear models in both unadjusted and adjusted models (e.g., age, gender, nasopharyngeal symptoms, chest radiographic results if available). Chest radiographic results were not used in the final analysis as most of the subjects did not have a chest x-ray completed and as the results were not binary, there was no grading scale to convert the reports by the radiologist into a metric that could be analyzed by the study team.

In total, 218 subjects participated in the clinical study and completed the COVD Smell Test. COVID-19 status was confirmed based on the results of the RT-PCR test linked to the subject's medical record. There were 47 subjects (21.6%) that tested positive for COVID-19 based on the results of the RT-PCR test obtained from their medical record. There were 79 subjects (36.2%) that tested negative for COVID-19, based on the results of the RT-PCR test obtained from their medical record. There were 92 subjects (42.2%) that were unable to link back to the subject's medical record to obtain the results of the RT-PCR and confirm their COVID-19 status. Data sets from the (n=47 COVID-19 positive) and (n=79 COVID-19 negative) subjects were analyzed. Data from the 92 subjects who completed the COVID Smell Test but were unable to link back to their medical records to confirm their COVID-19 status based on RT-PCR test is labeled as “Missing Outcome Data” in the following tables.

Table 2 below shows the demographics of subjects testing positive or negative for the COVID-19 virus by RT-PCR or missing COVID-19 test results. As shown below, the mean age of COVID-19 positive subjects is 37.5 years and 39.3 years in the COVID-19 negative subjects, which were not significantly different. The COVID-19 positive group included 27 (57%) females and the COVID-19 negative group included 46 females (58%), which were not significantly different. The missing outcome data for gender was due to gender not being fully recoverable from the electronic health record.

TABLE 2

| | COVID+ | COVID− | | Missing |
|---|---|---|---|---|
| | Patients | Patients | | Outcome Data |
| Parameter | (n = 47) | (n = 79) | p-value | (n = 92) |
| Age, yrs (SEM) | 37.5 (1.5) | 39.3 (1.5) | 0.16 | 38.4 (1.5) |
| % Sex - Female (n) | 57% (27) | 58% (46) | 0.42 | 68% (30) |

All enrolled subjects (n=218) answered the Symptom Tracking Survey (by selecting “Yes” or “No”) to questions 1-14, and 16, and, if applicable based on the branching logic design, additional information was provided for questions 15, 17, and 18 (see, e.g., Table 3 below). Table 3 below provides the number and percentage of subjects who answered ‘Yes’ to questions 1-14, and 16 in the column labeled ‘Question’. For question 14, “What is your temperature?” the result is presented in the table as a mean temperature based on the number of responses. For Question 16, “if yes: What was the result?” each indication, positive/negative/pending is presented in the table based on the number of responses to each indication. For question 17, “On what date was the COVID-19 test performed?” the result is presented in the table based as the number of responses providing a date. Data from the group of subjects (n=92) who successfully completed the COVID Smell Test but were unable to link the medical record to their COVID-19 status will be included in Table 3 labeled as “Missing Outcome Data.” Symptom scores were analyzed using a Chi-squared test (p-value reported) and Fisher's exact test. The presence of statistical significance (p <0.05) agreed between each analysis. The numbers and percentages are stratified by COVID-19 status.

TABLE 3

| | | | | Missing |
|---|---|---|---|---|
| | | | | Outcome Data |
| | COVID+ | COVID− | | (n = 92) |
| | Patients | Patients | | % of Missing |
| | (n = 47) | (n = 79) | | Outcome Data |
| | % of Yes | % of Yes | | (Yes) |
| Question | (n = yes) | (n = yes) | p-value | (n = yes) |
| 1. Do you currently | 17.02% | 12.66% | 0.50 | 8.70% |
| have a runny nose? | (n = 8) | (n = 10) | | (n = 8) |
| 2. Do you currently | 27.66% | 16.46% | 0.13 | 6.52% |
| have a cough? | (n = 13) | (n = 13) | | (n = 6) |
| 3. Do you currently | 21.28% | 15.19% | 0.38 | 10.87% |
| have a sore throat? | (n = 10) | (n = 12) | | (n = 10) |
| 4. Do you currently | 25.53% | 17.72% | 0.29 | 7.61% |
| have muscle aches? | (n = 12) | (n = 14) | | (n = 7) |
| 5. Do you currently | 8.51% | 11.39% | 0.61 | 5.43% |
| have shortness of | (n = 4) | (n = 9) | | (n = 5) |
| breath? | | | | |
| *6. Have you | 0.00% | 45.45% | 0.37 | 23.08% |
| experienced more | (n = 0) | (n = 10) | | (n = 6) |
| fatigue than usual? | | | | |
| 7. Are you currently | 38.30% | 6.33% | <0.0001 | 10.87% |
| lacking your sense | (n = 18) | (n = 5) | | (n = 10) |
| of smell? | | | | |
| 8. Is your current | 40.43% | 74.68% | 0.0001 | 69.57% |
| sense of smell what | (n = 19) | (n = 59) | | (n = 64) |
| it used to be? | | | | |
| 9. Is your current | 38.30% | 11.39% | 0.0004 | 16.30% |
| sense of taste | (n = 18) | (n = 9) | | (n = 15) |
| altered? | | | | |
| *Have you skipped | 0.00% | 31.82% | 0.50 | 23.08% |
| meals? | (n = 0) | (n = 7) | | (n = 6) |
| 10. Are you | 2.13% | 3.80% | 0.60 | 4.35% |
| currently smoking? | (n = 1) | (n = 3) | | (n = 4) |
| 11. Do odors cause | 8.51% | 6.33% | 0.65 | 9.78% |
| you to get sick, (eg., | (n = 4) | (n = 5) | | (n = 9) |
| asthma)? | | | | |
| 12. Do you currently | 2.13% | 6.33% | 0.28 | 5.43% |
| have a fever? | (n = 1) | (n = 5) | | (n = 5) |
| 13. Have you taken | 46.81% | 48.10% | 0.89 | 31.52 |
| your temperature? | (n = 22) | (n = 38) | | (n = 29) |
| 14. If yes, what is | 31.9% | 45.6% | 0.14 | 25.00% |
| your current | (97.73° F.) | (98.19° F.) | (0.07) | (98.12° F.) |
| temperature? (mean | (n = 15) | (n = 36) | | (n = 23) |
| temp) | | | | |
| *15. Have you been | 2.13% | 26.58% | — | 9.78% |
| tested for COVID-19? | (n = 1) | (n = 21) | | (n = 9) |
| *16. If yes: what was | — | — | — | — |
| the result? | | | | |
| Positive | 0% | 0% | — | 4.35% |
| | (n = 0) | (n = 0) | | (n = 4) |
| Negative | 0% | 12.66% | — | 4.35% |
| | (n = 0) | (n = 8) | | (n = 4) |
| Pending | 2.13% | 16.46% | — | 1.09% |
| | (n = 1) | (n = 13) | | (n = 1) |
| *17. If yes, on what | 2.13% | 26.58% | — | 9.78% |
| date was the COVID | (n = 1) | (n = 21) | | (n = 9) |
| test performed? | | | | |
| (number of subjects | | | | |
| that reported a date) | | | | |

Smell function was assessed in the Symptom Tracking Survey, question 7, “Are you currently lacking your sense of smell?” and question 8, “Is your current sense of smell what it used to be?” As shown, in Table 3, 38.3% (18 subjects) in the COVID-19 positive group responded “Yes” to question 7, “Are you currently lacking your sense of smell?”, whereas only 6.3% (5 subjects) in the COVID-19 negative group responded “Yes” (p <0.0001). For question 8, “Is your current sense of smell what it used to be?”, 40.4% (19 subjects) in the COVID-19 positive group responded “Yes” whereas 74.7% (59 subjects) in the COVID-19 negative group responded “Yes” (p =0.0001).

These results show that subjects who self-reported lacking sense of smell (38.3% in the COVID-19 positive group) or smell is not what it used to be (40.4% in the COVID-19 positive group) are experiencing olfactory deficits and loss of smell is a useful predictor of COVID-19 status. The sense of taste is an amalgam of olfactory input, taste receptors on the tongue, and texture, with the olfactory component comprising 70%. Taste alteration may be linked to smell loss with COVID-19 positive subjects relative to COVID-19 negative individuals. In this clinical study, 38.3% (18 subjects) in the COVID-19 positive group responded “Yes” to question 9, “Is your current sense of taste altered?” whereas only 11.4% (9 subjects) in the COVID-19 negative group answered “Yes.” This result (p value =0.0004) implies that taste alteration can also be a useful feature in predicting COVID-19 status since a significantly higher proportion of individuals that are COVID-19 positive have self-reported taste alteration.

In addition, results show the absolute difference in percentages of ‘Yes’ answers for COVID-19 positive patients and ‘Yes’ answers for COVID-19 negative patients. The larger this difference, the more discriminating the variable. In question 7, “Are you currently lacking your sense of smell” there is a large absolute difference of 32% percent between the two groups. Additionally, the percentage for COVID-19 positive is 6.05 times that of COVID-19 negative (the relative difference). This demonstrates there is already signal in this variable, since if a future user self-reports smell alteration and they are sampled from a similar population as these data represent, it would be expected that they would have a higher likelihood of being COVID-19 positive. Of note, the presence of fever and the recorded temperatures did not associate with COVID-19 positive or COVID-19 negative status in this study.

Based on the results from the clinical study, the symptoms from the Symptom Tracking Survey that were selected by the machine learning algorithm for prediction of COVID-19 status include: “Are you currently lacking your sense of smell?”, “Is your current sense of smell what it used to be?”, and “Is your current sense of taste altered?”. These variables have the largest differences between the COVID-19 positive and COVID-19 negative groups as measured by the Symptom Tracking Survey. Other symptoms such as cough and muscle aches were not selected.

All enrolled subjects (n=218) completed the intensity test of Test 1. Table 4 below provides the mean intensity score reported by each subject for each of the three odors. Odor intensity was assessed by requiring subjects to smells each of the three odors and then enter a score to describe the intensity of how each of the three odors smell on a scale from 0-10 (0=not detectable and 10=strongest detectable). This data was stratified by COVID-19 status.

TABLE 4

| | | | | | | Missing | |
|---|---|---|---|---|---|---|---|
| | COVID-19 | | COVID-19 | | | Outcome | |
| | Positive | | Negative | | | Data (MOD) | |
| | (n = 47) | | (n = 79) | | | (n = 92) | |
| Intensity | Mean | SEM | Mean | SEM | p-Value | Mean | SEM |
| Odor 1 | 5.70 | 0.43 | 7.24 | 0.27 | 0.003 | 6.42 | 0.30 |
| Odor 2 | 6.02 | 0.49 | 8.09 | 0.21 | 0.0009 | 7.22 | 0.31 |
| Odor 3 | 5.34 | 0.50 | 7.00 | 0.25 | 0.015 | 6.14 | 0.33 |
| Average | 5.69 | 0.43 | 7.44 | 0.20 | 0.0017 | 6.59 | 0.28 |
| Intensity | | | | | | | |

Differences in distributions of intensity ratings between COVID-19 positive and COVID-19 negative groups were analyzed using the nonparametric Mann-Whitney Test. The subjects with a documented COVID-19 infection recorded statistically significantly lower intensities for each of the three odors. The distribution difference of the combined intensity ratings was also highly statistically significant.

All enrolled subjects (n=218) also completed the odor identification tests of Test 1. Odor percept identification was assessed by requiring subjects to correctly identify the name of each of the three odors presented: Odors 1(grape), 2 (smoke), and 3 (soap). The following table (Table 5) provides the number and percentages of subjects who correctly identified the name of the odor presented, stratified by COVID-19 status. Differences in the identification scores between COVID-19 positive and COVID-19 negative groups were analyzed using a Chi-squared (p value reported) and Fisher's Exact test.

TABLE 5

| | | | | | | Missing Outcome | |
|---|---|---|---|---|---|---|---|
| | COVID Positive | | COVID Negative | | | Data (MOD) | |
| Odor Percept | (n = 47) | | (n = 79) | | | (n = 92) | |
| Identification | Mean | SEM | Mean | SEM | p-Value | Mean | SEM |
| Odor 1 (Grape) | 37 | 78.72% | 68 | 86.08% | 0.28 | 73 | 79.35% |
| Odor 2 (Smoke) | 34 | 72.34% | 74 | 93.67% | 0.0009 | 72 | 78.26% |
| Odor 3 (Soap) | 35 | 74.47% | 75 | 94.94% | 0.0008 | 72 | 78.26% |
| Average | | 75% | | 92% | 0.0025 | | 79% |
| Identification (SEM) | | (5%) | | (2%) | | | (3%) |

Both percepts of Odors 2 and 3 were identified significantly worse in subjects with a documented COVID-19 infection by RT-PCR (COVID-19 positive). Odor 1 was not significantly different, with decreased performance in the COVID-19 negative group.

All enrolled subjects (n=218) also completed Test 2, which consisted of odor discrimination tests. Discrimination was assessed by requiring subjects to distinguish odors to smell a row of three odors and correctly identify which of the three odors was different from the other two. This was repeated in three separate discrimination trials. Differences in the discrimination scores between COVID-19 positive and COVID-19 negative group were analyzed using a Chi-squared or Fisher's Exact test. In each of the 3 discrimination trials, a statistically significant greater percentage of the COVID-19 negative group was able to correctly make the discrimination compared to the COVID-19 positive group as shown below in Table 6.

TABLE 6

| | | | | | | Missing Outcome | |
|---|---|---|---|---|---|---|---|
| | COVID Positive | | COVID Negative | | | Data (MOD) | |
| | (n = 47) | | (n = 79) | | | (n = 92) | |
| Discrimination | Mean | SEM | Mean | SEM | p-Value | Mean | SEM |
| Discrimination trial 1 | 37 | 78.72% | 74 | 93.67% | 0.012 | 79 | 85.87% |
| Discrimination trial 2 | 30 | 63.83% | 68 | 86.08% | 0.0037 | 78 | 84.78% |
| Discrimination trial 3 | 35 | 74.47% | 70 | 88.61% | 0.039 | 71 | 77.17% |
| Average | | 72% | | 89% | 0.007 | | 83% |
| Discrimination (SEM) | | (5%) | | (2%) | | | (0.3%) |

Based on the statistically significant results of the clinical study collecting both symptomatic data from the Symptom Tracking Survey and smell assessment data from the Odor Intensity, Identification, and Discrimination from the COVID Smell Test, for patients with validated COVID-19 RT-PCR status, multivariable logistic regression was used to predict overall COVID-19 status defined by RT-PCR.

In a first approach to generate an algorithm that could be embedded into a container, for the cohort of 126 patients with COVID-19 status (n=47 COVID-19 positive and n=79 COVID-19 negative), the variables were age (range 18-78) both linear and as a quadratic to account for age-associated changes in smell loss, sex, lost smell symptom, smell normal symptom, taste altered symptom, average intensity score, average identification score, average discrimination score, number of identification and discrimination trials that were missed, age interaction with average identification, to account for smell loss due to pre-clinical issues, and time of testing relative to start of the study, to account for the changing positivity rate of RT-PCR results.

After backwards elimination, the lost smell symptom, the number missing out of the six identification and discrimination trials, and days of the study survived, indicating non-equivalent sampling of COVID-19 positivity due to flattening the curve during the summer.

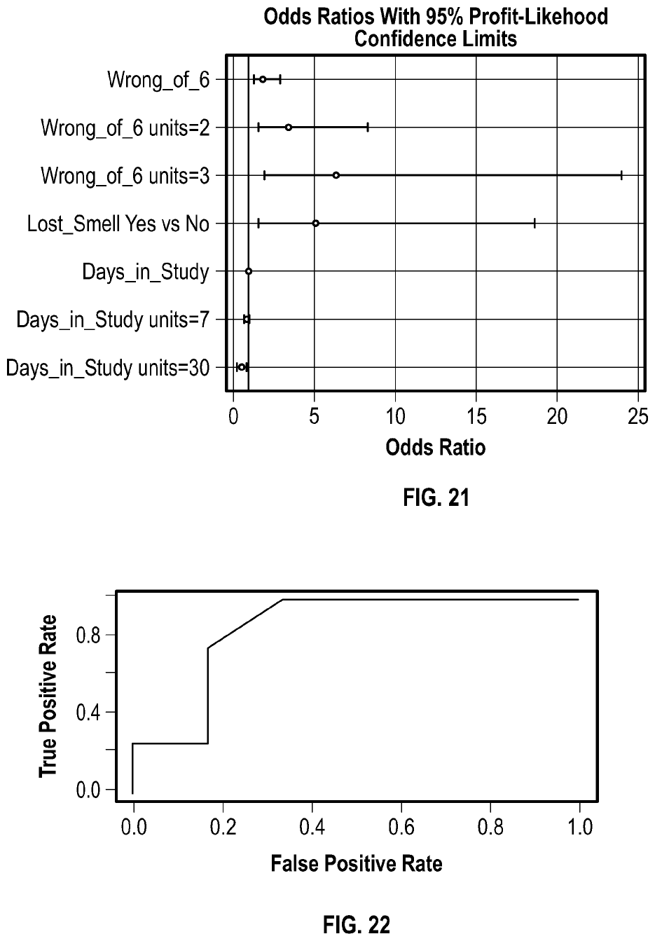

The final model converged with an overall p value of <0.0001, and the p values for lost smell, number of identification and discrimination missed, and days in the study were 0.0082, 0.0035, and 0.0094, respectively. The area under the curve of the ROC plot was 0.7712. The odds ratios are shown in FIG. 21. In FIG. 21, results with error bars (95% confidence intervals) for each instantiation of each variable. For the Lost Smell symptom and the 2 missed trials (Wrong_of_6 units=2) and 3 missed trials (Wrong_of_6 units=3), the Days in Study were set at the mean value (36.73 days). The lost smell symptom (OR=5.1x) has been associated with COVID-19 infection. The missed (responded incorrectly) identification and discrimination question was also significant at the threshold of two missed questions (OR=3.4x) and three missed questions (OR=6.3x).

Importantly, there was no interaction between the lost smell variable and incorrectly answering two or more questions on the identification and discrimination tests (p=0.65), indicating that poor performance on the objective test was independent of one's subjective impression about smell loss. Also, the elapsed time of the study also showed significant diminished risk (OR=0.51x) of having an RT-PCR test positive for COVID-19 30 days after the study began, while the curve was flattening in this study population.

In a second approach to generate an algorithm (e.g., algorithm 550, FIG. 15) that could be embedded into a container so that the result could be delivered in real time on the app, all symptom responses and smell test data including feature engineered variables—missing two or more Identification and Discrimination questions, missing one Identification or Discrimination with an Intensity of less than predetermined threshold (in this study, 6)—were incorporated in a lasso procedure using generalized linear regression to predict COVID-19 status. The machine learning approach included both subjective symptomatic and objective smell test data in the generation of the predictor that was selected to optimize interpretability. The predictor returned the three symptoms (lost smell, smell normal, and taste altered) as well as missing two or more questions, Intensity of odor 2, and Identification of odor 2 as contributing to the prediction. The algorithm included using logistic regression to determine weighting coefficients for each of the symptom questionnaire responses as well as each of the odor tests, including the engineered variables. Those weighting factors could then be used to discern which tests were statistically significant to predict the infection status of a subject. Those same weighting coefficients were then used to generate a cumulative test score (e.g., multiplying the result of the individual tests by the weighting coefficient, and adding those results together to generate a cumulative score), which was then used to determine or predict a risk status for infection with a comparison against a predetermined threshold.

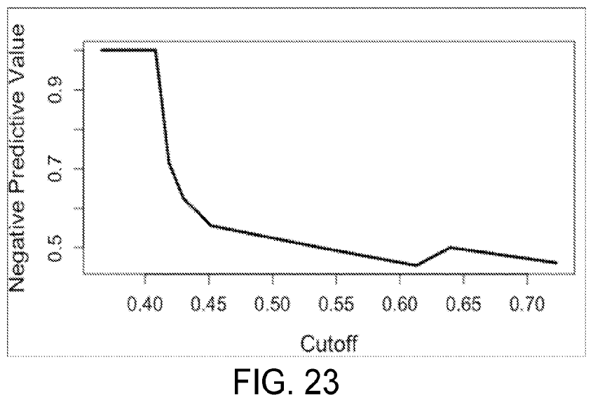

Using the algorithm developed from data from the first 74 patients with a documented COVID-19 status, the algorithm performance achieved a sensitivity of 100% and a specificity of 67% predicting COVID-19 status defined by the NP RT-PCR assay in the next 14 patients (FIG. 22). As illustrated in FIG. 22, the AUC for the machine learning algorithm was 0.85 in this test set. As illustrated in FIG. 23, the negative predictive value was 100% at a cutoff of 0.40.

As a result of the clinical study data, the algorithm has been containerized into the web-based app. Based on the clinical study results and the algorithm, binary results can now be provided to the subject via the web-based app at the end of the COVID Smell Test in real time to each individual taking the test. After completing the COVID Smell Test, the individual is provided with a result from the app and is informed that their sense of smell is normal or abnormal.

If the individual's sense of smell is determined to be normal the return results are shown on the screen as GREEN (low-risk): Normal. Your sense of smell appears to be normal. If the individual's sense of smell is determined to be abnormal the return results are shown on the screen as RED (high-risk): Abnormal. Your sense of smell appears to be abnormal. Please consult a healthcare professional. Loss of smell has been identified as one sign associated with positive COVID-19 infection.

The COVID Smell Test demonstrated a robust association of olfactory dysfunction based on the results of the Symptom Tracking Survey and the olfactory tests of Intensity, Identification, and Discrimination with infection by COVID-19 (model convergence with p <0.0001). Based on the results of the Symptom Tracking Survey the three symptoms below contributed to the prediction of COVID-19 positive results confirmed by RT-PCR results in the medical record of each subject (p <0.0082). One, “are you currently lacking your sense of smell?” (p <0.0001). Two, “is your current sense of smell what it used to be?” (p=0.0001). And, three, “is your current sense of taste altered?” (p=0.0004). Patients with validated COVID-19 positive results confirmed by RT-PCR test performed worse on the odor Intensity ratings (p <0.0017), odor percept Identification (p <0.0025), and odor Discrimination (p <0.0007).

In multivariate analysis, incorrect responses on objective odor tests of both Identification and Discrimination added more predictive power than Identification alone (p <0.0035). Incorrect responses survived backward elimination together with asking the subject about their impression of smell loss in the Symptom Tracking Survey (p <0.0082). Our results demonstrate the potential for combining both self-reported symptoms by the Symptom Tracking Survey and objective olfactory dysfunction measured by the odor tests provide a robust predictor of COVID-19 infection that may benefit the intended patient population.

These study results demonstrate that detecting an olfactory deficit increases the odds ratio of having COVID-19 infection by 5-7-fold. Therefore, use of the COVID Smell Test with immediate results by the general population may quickly identify individuals who are at risk of COVID-19 infection so that they may take immediate action to protect themselves and others by self-quarantining and consulting with a healthcare professional. Moreover, the high negative predictive value of the COVID Smell Test provides important pre-test probability information that can potentially be combined with pooling strategies to reduce the number of RT-PCRs needed for screening populations to meet the demand of the more than 200 million molecular tests that are needed on a monthly basis. The COVID Smell Test provides a cost-effective, easy to use screening test that can be rapidly scaled up to produce millions of tests in a few weeks addressing a significant unmet medical need with the ongoing public health emergency of the COVID-19 pandemic.

At present, there are no olfactory tests that have been cleared, approved, or authorized for emergency use by FDA to screen for olfactory loss as a sign of potential COVID-19 infection. Results of this study demonstrate that the COVID Smell Test introduces a significant benefit to the general population to provide a reliable test in an easy to use, convenient, and rapid manner so that patients can screen themselves and take appropriate actions to reduce exposure to others or seek care from a medical professional for confirmatory diagnosis and/or treatment.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

Thus, while the invention has been described in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A test kit comprising:
an odor proctor configured to deliver an odor from among a plurality of odors;
an application on a computing device comprising instructions that, when executed by a processor running on the computing device, cause the computing device to:
generate a user interface to be displayed on the computing device;
receive, via the user interface, a user input for a smell test comprising at least one of an odor intensity test, an odor identification test, or an odor discrimination test;
calculate, based on the user input for the smell test, a score for the smell test; and
using the score for the smell test, generate a report indicating a user being affected with symptoms associated with COVID-19.

2. The test kit of claim 1, wherein the odor proctor includes a printed test card.

3. The test kit of claim 2, wherein each of the plurality of odors on the printed test card include a cover thereon configured to encapsulate a scent of the plurality of odors.

4. The test kit of claim 1, wherein the odor proctor is configured to be disposable.

5. The test kit of claim 1, wherein the computing device is one of a personal computer, tablet, or smart phone.

6. The test kit of claim 1, wherein the instructions include steps for administering the odor intensity test that cause the computing device to:
instruct the user of the computing device to smell an odor on the odor proctor;
generate an odor intensity user interface comprising an intensity rating function;
receive from the user, via the intensity rating function, an intensity rating; and
calculate a score for the odor intensity.

7. The test kit of claim 6, test, wherein if the user inputs a high intensity rating, the score is increased, and if the user inputs a low intensity rating, the score is decreased.

8. The test kit of claim 1, wherein the instructions include steps for administering the odor identification test that cause the computing device to:
instruct the user of the computing device to smell an odor on the odor proctor;
generate an odor identification user interface comprising an odor selection function;
receive from the user, via the odor selection function, a selection of an odor identification;
query a database to identify a correct response for the selection; and
calculate a score for the odor identification test.

9. The test kit of claim 8, wherein if the selection matches the correct response, the score is increased, and if the selection does not match the correct response, the score is decreased.

10. The test kit of claim 1, wherein the instructions include steps for administering the odor discrimination test that cause the computing device to:
instruct the user of the computing device to smell a first odor, a second odor, and a third odor on the odor proctor;
generate an odor discrimination user interface comprising an odor discrimination function;
receive from the user, via the odor discrimination function, a selection of one of the first odor, the second odor, or the third odor;
query a database to identify a correct response for the selection; and
calculate a score for the odor discrimination test.

11. The test kit of claim 10, wherein if the selection matches the correct response, the score is increased, and if the selection does not match the correct response, the score is decreased.

12. A method of administering a smell test for assessment of a user being affected by symptoms associated with SARS coronaviruses or COVID-19, the method comprising:
generating, by a computing device, a user interface to be displayed on the computing device;
instructing, by the computing device via the user interface, a user to utilize an odor proctor configured to deliver an odor from among a plurality of odors;
receiving, by the computing device via the user interface, a user input for a smell test;
calculating, by the computing device, a score for the smell test; and
based on the score for the smell test, generating a report indicating the user being affected with symptoms associated identifying a user as high risk for being infected with SARS coronaviruses or COVID-19.

13. The method of claim 12, wherein the method further comprises identifying, based on the score for the smell test, the user as high risk for being infected with SARS coronaviruses or COVID-19.

14. The method of claim 12, wherein the smell test includes at least one of an odor intensity test, an odor identification test, or an odor discrimination test.

15. The method of claim 14, wherein steps for administering the odor intensity test comprises:
instructing, by the computing device, the user of the computing device to smell an odor on the odor proctor;
generating, by the computing device, an odor intensity user interface comprising an intensity rating function;
receiving from the user, via the intensity rating function, an intensity rating; and
calculating, by the computing device, a score for the odor intensity tests, wherein if the user inputs a high intensity rating, the score is increased, and if the user inputs a low intensity rating, the score is decreased.

16. The method of claim 15, wherein the odor intensity test comprises a first odor intensity test, a second odor intensity test, and a third odor intensity test.

17. The method of claim 16, wherein steps for administering the odor identification test comprises:
instructing, by the computing device, the user of the computing device to smell an odor on the odor proctor;
generating, by the computing device, an odor identification user interface comprising an odor selection function;
receiving from the user, via the odor selection function, a selection of an odor identification;
querying a database to identify a correct response for the selection; and
determining if the selection matches the correct response.

18. The method of claim 17, wherein the odor identification test comprises a first odor identification test, a second odor identification test, and a third odor identification test.

19. The method of claim 18, wherein the steps for administering the odor discrimination test comprises:
instructing, via the computing device, the user of the computing device to smell a first odor, a second odor, and a third odor on the odor proctor;
generating, by the computing device, an odor discrimination user interface comprising an odor discrimination function;
receiving from the user, via the odor discrimination function, a selection of one of the first odor, the second odor, or the third odor;
query a database to identify a correct response for the selection; and
determining if the selection matches the correct response.

20. The method of claim 19, wherein the odor discrimination test comprises a first odor discrimination test, a second odor discrimination test, and a third odor discrimination test.

21. The method of claim 12, further comprising:
generating, via the computing device, a symptom questionnaire interface; and
receiving from the user, via the symptom questionnaire interface, a user input of binary responses to a plurality of COVID-19 symptom-based questions.

22. The method of claim 12, further comprising:
comparing, by the computing device, the score for the smell test relative to a predetermined threshold, wherein the predetermined threshold is determined by a machine learning algorithm based on a plurality of previously-entered smell test performance data.

23. The method of claim 13, wherein, upon the identification of the user as high risk for being infected with COVID-19, disposing of the odor proctor.
